(19) United States
(12) Patent Application Publication
(10) Pub. No.: US 2010/0297722 A1
(43) Pub. Date: Nov. 25, 2010

(54) TRANSGENIC MOSS PRODUCING TERPENOIDS

(75) Inventors: Aldwin Macaraig Anterola, Carbondale, IL (US); Ralph Stephen Quatrano, St. Louis, MO (US); Pierre-Francois Perroud, St. Louis, MO (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignees: BOARD OF TRUSTEES OF SOUTHERN ILLINOIS UNIVERSITY, Carbondale, IL (US); WASHINGTON UNIVERSITY, St. Louis, MO (US)

(21) Appl. No.: 12/783,952

(22) Filed: May 20, 2010

Related U.S. Application Data
(60) Provisional application No. 61/180,123, filed on May 20, 2009.

(51) Int. Cl. C12P 5/02; C12N 15/82; C12N 5/10

(52) U.S. Cl. 435/167; 435/468; 435/419

(57) ABSTRACT

The present invention generally relates to transgenic moss. One aspect of the invention provides a transgenic moss cell that produces or accumulates a terpenoid compound. Another aspect of the invention provides for methods of producing a terpenoid compound through culturing of the transgenic moss.

### CROSS-REFERENCE TO RELATED APPLICATIONS

This application claims priority from U.S. Provisional Application Ser. No. 61/180,123, filed on May 20, 2009, which is incorporated herein by reference in its entirety.

### STATEMENT REGARDING FEDERALLY SPONSORED RESEARCH OR DEVELOPMENT

This invention was made in part with Government support under National Institutes of Health Grant 1 R15 CA139416-01. The Government has certain rights in the invention.

### INCORPORATION-BY-REFERENCE OF MATERIAL SUBMITTED ON A COMPACT DISC

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to transgenic moss, more specifically transgenic moss that produces or accumulates a terpenoid compound.

### BACKGROUND

Various taxoids are promising therapeutic agents. Paclitaxel (commonly known as Taxol™) is a widely prescribed anticancer agent originally isolated as a bioactive component from the bark of Pacific yew (Taxus brevifolia) and later structurally defined as a very complex multi-substituted taxane diterpenoid. Naturally occurring plant taxoids are generally present in extremely low levels. Chemical methods to totally synthesize paclitaxel exist but are not suitable for large-scale commercial production of the drug. Paclitaxel is currently manufactured from an advanced paclitaxel precursor 10-deacetylbaccatin III, extracted from needles of European yew (Taxus baccata); but yields from yew are highly variable and affected by environmental conditions and yew cultivation requires substantial land area, long time prior to harvest, intensive labor, and additional costs for extraction and semisynthesis of the drug.

Liquid cell suspension cultures of Taxus species can be induced to produce paclitaxel but reproducibility of various induction treatments from one cell line to another have yet to be demonstrated, and Taxus cells (like other cultured plant cells) undergo epigenetic and mutational changes while in culture which can change their totipotency and ability to produce paclitaxel.

Endophytic fungi living inside Taxus brevifolia are known to produce paclitaxel but do not grow well in culture and produce only minute amounts of paclitaxel. Various approaches have been used to overexpress paclitaxel biosynthetic genes in endophytic fungi to improve paclitaxel production but slow growth of the fungi host cells impose limitations on productivity (see generally, Heinig and Jennewein (2009) African J of Biotech 8(8) 1370-1385).

Faster-growing heterologous systems such as bacteria and yeasts have been used to overexpress paclitaxel biosynthetic genes but neither yeast nor bacteria has been successful so far in producing advanced paclitaxel precursors.

Higher plants used as heterologous hosts for overexpression of taxadiene synthase may result in increased taxoids but grow more slowly than wild type, presumably due to disruption in gibberellin biosynthesis.

### SUMMARY OF THE INVENTION

Among the various aspects of the present invention is the provision of compositions and processes for production of terpenoids from metabolically engineered moss. Such an engineered moss can accumulate a target terpenoid compound, such as a taxoid or intermediate thereof, without substantially deleterious phenotypic consequences.

One aspect provides a moss cell comprising at least a first heterologous nucleic acid molecule, wherein expression of the heterologous nucleic acid molecule in the cell results in production of at least one target terpenoid compound. The at least one target terpenoid compound can be taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin III; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.

In some embodiments, the moss cell comprises a first heterologous nucleic acid molecule comprising a nucleotide sequence encoding a taxadiene synthase and the moss cell produces or accumulates at least taxa-4(5),11(12)-diene.

In some embodiments, the moss cell comprises a first heterologous nucleic acid molecule comprising a polynucleotide encoding a taxadiene synthase; and second heterologous nucleic acid molecule comprising a polynucleotide encoding a taxadiene 5α-hydroxylase; and the moss cell produces or accumulates at least taxadiene-5-ol.

In some embodiments, the moss cell comprises a first heterologous nucleic acid molecule comprising a polynucleotide encoding a taxadiene synthase; a second heterologous nucleic acid molecule comprising a polynucleotide encoding a taxadiene-5α-hydroxylase; a third heterologous nucleic acid molecule comprising a polynucleotide encoding a taxadiene-5α-ol-acetyl transferase; and a fourth heterologous nucleic acid molecule comprising a polynucleotide encoding a taxane-13α-hydroxylase; and the moss cell produces or accumulates at least taxadiene-5α-acetoxy-13β-ol.

In some embodiments, the moss cell comprises a first heterologous nucleic acid molecule comprising a polynucleotide encoding a taxadiene synthase; a second heterologous nucleic acid molecule comprising a polynucleotide encoding a taxadiene-5α-hydroxylase; and a third heterologous nucleic acid molecule comprising a polynucleotide encoding a taxane-13α-hydroxylase; and the moss cell produces or accumulates at least taxadiene-5α,13α-diol.

In some embodiments, the moss cell comprises a first heterologous nucleic acid molecule comprising a polynucleotide encoding a taxadiene synthase; a second heterologous nucleic acid molecule comprising a polynucleotide encoding a taxadiene-5α-hydroxylase; a third heterologous nucleic acid molecule comprising a polynucleotide encoding a taxadiene-5α-ol-acetyl transferase; and a fourth heterologous nucleic acid molecule comprising a polynucleotide encoding a taxane-10β-hydroxylase; and the moss cell produces or accumulates at least taxadiene-5α-acetoxy-10β-ol.

In some embodiments, the moss cell comprises a first heterologous nucleic acid molecule comprising a polynucleotide encoding a taxadiene synthase; a second heterologous nucleic acid molecule comprising a polynucleotide encoding a taxadiene-5α-hydroxylase; a third heterologous nucleic acid molecule comprising a polynucleotide encoding a taxadiene-5α-ol-acetyl transferase; a fourth heterologous nucleic acid molecule comprising a polynucleotide encoding a taxane-10β-hydroxylase; a fifth heterologous nucleic acid molecule comprising a polynucleotide encoding a taxane-13α-hydroxylase; a sixth heterologous nucleic acid molecule comprising a polynucleotide encoding a taxoid-9α-hydroxylase; a seventh heterologous nucleic acid molecule comprising a polynucleotide encoding a taxoid-2α-hydroxylase; an eighth heterologous nucleic acid molecule comprising a polynucleotide encoding a taxoid-7β-hydroxylase; a ninth heterologous nucleic acid molecule comprising a polynucleotide encoding a 2α-hydroxytaxane 2-O-benzoyltransferase; a tenth heterologous nucleic acid molecule comprising a polynucleotide encoding a taxoid C1β-hydroxylase; and a eleventh heterologous nucleic acid molecule comprising a polynucleotide encoding a taxoid C4β, C20-epoxidase; and the moss cell produces or accumulates at least 10-deacetylbaccatin III.

In some embodiments, the moss cell comprises a first heterologous nucleic acid molecule comprising a nucleotide sequence encoding an abietadiene synthase; and the moss cell produces or accumulates at least abietadiene.

In some embodiments, the moss cell comprises a first heterologous nucleic acid molecule comprising a nucleotide sequence encoding an abietadiene synthase; and a second heterologous nucleic acid molecule comprising a nucleotide sequence encoding an abietadienol/abietadienal oxidase; and the moss cell produces or accumulates at least abietic acid.

In some embodiments, the moss cell comprises a first heterologous nucleic acid molecule comprising a nucleotide sequence encoding an ent-copalyl diphosphate synthase; a second heterologous nucleic acid molecule comprising a nucleotide sequence encoding an ent-Kaurene synthase; a third heterologous nucleic acid molecule comprising a nucleotide sequence encoding an ent-Kaurene oxidase; and a fourth heterologous nucleic acid molecule comprising a nucleotide sequence encoding an kaurenoic acid 13-hydroxylase; and the moss cell produces or accumulates at least steviol.

In some embodiments, the moss cell comprises a first heterologous nucleic acid molecule comprising a nucleotide sequence encoding an ent-copalyl diphosphate synthase; a second heterologous nucleic acid molecule comprising a nucleotide sequence encoding an ent-Kaurene synthase; a third heterologous nucleic acid molecule comprising a nucleotide sequence encoding an ent-Kaurene oxidase; a fourth heterologous nucleic acid molecule comprising a nucleotide sequence encoding an kaurenoic acid 13-hydroxylase; and a fifth heterologous nucleic acid molecule comprising a nucleotide sequence encoding an UDP-glycosyltransferase (UGT) UGT85C2; and the moss cell produces or accumulates at least steviolmonoside.

In some embodiments, the moss cell comprises a first heterologous nucleic acid molecule comprising a nucleotide sequence encoding an ent-copalyl diphosphate synthase; a second heterologous nucleic acid molecule comprising a nucleotide sequence encoding an ent-Kaurene synthase; a third heterologous nucleic acid molecule comprising a nucleotide sequence encoding an ent-Kaurene oxidase; a fourth heterologous nucleic acid molecule comprising a nucleotide sequence encoding an kaurenoic acid 13-hydroxylase; a fifth heterologous nucleic acid molecule comprising a nucleotide sequence encoding an UDP-glycosyltransferase (UGT) UGT85C2; and a sixth heterologous nucleic acid molecule comprising a nucleotide sequence encoding a UGT74G1; and the moss cell produces or accumulates at least stevioside.

In some embodiments, the moss cell comprises a first heterologous nucleic acid molecule comprising a nucleotide sequence encoding an ent-copalyl diphosphate synthase; a second heterologous nucleic acid molecule comprising a nucleotide sequence encoding an ent-Kaurene synthase; a third heterologous nucleic acid molecule comprising a nucleotide sequence encoding an ent-Kaurene oxidase; a fourth heterologous nucleic acid molecule comprising a nucleotide sequence encoding an kaurenoic acid 13-hydroxylase; a fifth heterologous nucleic acid molecule comprising a nucleotide sequence encoding an UDP-glycosyltransferase (UGT) UGT85C2; a sixth heterologous nucleic acid molecule comprising a nucleotide sequence encoding a UGT74G1; and a seventh heterologous nucleic acid molecule comprising a nucleotide sequence encoding a UGT76G1; and the moss cell produces or accumulates at least rebaudioside A.

In some embodiments, the moss cell comprises a first heterologous nucleic acid molecule comprising a nucleotide sequence encoding an ent-copalyl diphosphate synthase; a second heterologous nucleic acid molecule comprising a nucleotide sequence encoding an ent-Kaurene synthase; a third heterologous nucleic acid molecule comprising a nucleotide sequence encoding an ent-Kaurene oxidase; and the moss cell produces or accumulates at least kaurenoic acid.

In some embodiments, the moss cell comprises a first heterologous nucleic acid molecule comprising a nucleotide sequence encoding a CBT-ol cyclase; and a second heterologous nucleic acid molecule comprising a nucleotide sequence encoding a CYP71D16; and the moss cell produces or accumulates at least a cembranoid.

In some embodiments, the moss cell comprises a first heterologous nucleic acid molecule comprising a nucleotide sequence encoding a syn-copalyl diphosphate synthase; and a second heterologous nucleic acid molecule comprising a nucleotide sequence encoding a syn-pimaradiene synthase; and the moss cell produces or accumulates at least momilactones A and B.

In some embodiments, the moss cell comprises a first heterologous nucleic acid molecule comprising a nucleotide sequence encoding a ent-Copalyl diphosphate synthase; and a second heterologous nucleic acid molecule comprising a nucleotide sequence encoding a ent-sandaracopimaradiene synthase; and the moss cell produces or accumulates at least one of oryzalexins A-F.

In some embodiments, the moss cell comprises a first heterologous nucleic acid molecule comprising a nucleotide sequence encoding a syn-copalyl diphosphate synthase; and a second heterologous nucleic acid molecule comprising a nucleotide sequence encoding a syn-stemarene synthase; and the moss cell produces or accumulates at least oryzalexin S.

In some embodiments, the moss cell comprises a first heterologous nucleic acid molecule comprising a nucleotide sequence encoding a ent-Copalyl diphosphate synthase; and a second heterologous nucleic acid molecule comprising a nucleotide sequence encoding a ent-cassadiene synthase; and the moss cell produces or accumulates at least one of phytocassanes A-E.

In some embodiments, the moss cell comprises a nucleotide sequence encoding a taxadiene synthase selected from the group consisting of: a nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17, or at least about 90% sequence identity to any one of these sequences encoding a polypeptide having taxadiene synthase activity, or a complementary sequence thereto; a nucleotide sequence encoding a polypeptide having SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18, or at least about 90% sequence identity to any one of these sequences having taxadiene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxadiene synthase activity.

In some embodiments, the moss cell comprises a nucleotide sequence encoding a taxadiene 5α-hydroxylase selected from the group consisting of: a nucleotide sequence of SEQ ID NO: 19 or SEQ ID NO: 21, or at least about 90% sequence identity to any one of these sequences encoding a polypeptide having taxadiene 5α-hydroxylase activity, or a complementary sequence thereto; a nucleotide sequence encoding a polypeptide having SEQ ID NO: 20 or SEQ ID NO: 22, or at least about 90% sequence identity to any one of these sequences having taxadiene 5α-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxadiene 5α-hydroxylase activity.

In some embodiments, the moss cell comprises a nucleotide sequence encoding a taxadiene-5α-ol-acetyl transferase selected from the group consisting of: a nucleotide sequence of SEQ ID NO: 23, SEQ ID NO: 25, or SEQ ID NO: 201, or at least about 90% sequence identity to any one of these sequences encoding a polypeptide having taxadiene-5α-ol-acetyl transferase activity, or a complementary sequence thereto; a nucleotide sequence encoding a polypeptide having SEQ ID NO: 24, SEQ ID NO: 26, or SEQ ID NO: 202, or at least about 90% sequence identity to any one of these sequences having—taxadiene-5α-ol-acetyl transferase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxadiene-5α-ol-acetyl transferase activity.

In some embodiments, the moss cell comprises a nucleotide sequence encoding a taxane-13α-hydroxylase selected from the group consisting of: a nucleotide sequence of SEQ ID NO: 27, SEQ ID NO: 29, or SEQ ID NO: 31, or at least about 90% sequence identity to any one of these sequences encoding a polypeptide having taxane-13α-hydroxylase activity, or a complementary sequence thereto; a nucleotide sequence encoding a polypeptide having SEQ ID NO: 28, SEQ ID NO: 30, or SEQ ID NO: 32, or at least about 90% sequence identity to any one of these sequences having taxane-13α-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxane-13α-hydroxylase activity.

In some embodiments, the moss cell comprises a nucleotide sequence encoding a taxane-10β-hydroxylase selected from the group consisting of: a nucleotide sequence of SEQ ID NO: 33, SEQ ID NO: 35, SEQ ID NO: 37, or SEQ ID NO: 39, or at least about 90% sequence identity to any one of these sequences encoding a polypeptide having taxane-10β-hydroxylase activity, or a complementary sequence thereto; a nucleotide sequence encoding a polypeptide having SEQ ID NO: 34, SEQ ID NO: 36, SEQ ID NO: 38, or SEQ ID NO: 40, or at least about 90% sequence identity to any one of these sequences having taxane-10β-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxane-10β-hydroxylase activity.

In some embodiments, the moss cell comprises a nucleotide sequence encoding a taxoid-2α-hydroxylase selected from the group consisting of: a nucleotide sequence of SEQ ID NO: 41 or SEQ ID NO: 43, or at least about 90% sequence identity to any one of these sequences encoding a polypeptide having taxoid-2α-hydroxylase activity, or a complementary sequence thereto; a nucleotide sequence encoding a polypeptide having SEQ ID NO: 42 or SEQ ID NO: 44, or at least about 90% sequence identity to any one of these sequences having taxoid-2α-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxoid-2α-hydroxylase activity.

In some embodiments, the moss cell comprises a nucleotide sequence encoding a taxoid-7β-hydroxylase selected from the group consisting of: a nucleotide sequence of SEQ ID NO: 45 or SEQ ID NO: 47, or at least about 90% sequence identity to any one of these sequences encoding a polypeptide having taxoid-7β-hydroxylase activity, or a complementary sequence thereto; a nucleotide sequence encoding a polypeptide having SEQ ID NO: 46 or SEQ ID NO: 48, or at least about 90% sequence identity to any one of these sequences having taxoid-7β-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having taxoid-7β-hydroxylase activity.

In some embodiments, the moss cell comprises a nucleotide sequence encoding a 2α-hydroxytaxane 2-O-benzoyltransferase selected from the group consisting of: a nucleotide sequence of SEQ ID NO: SEQ ID NO: 49, SEQ ID NO: 51, SEQ ID NO: 53, SEQ ID NO: 55, SEQ ID NO: 57, or SEQ ID NO: 59, or at least about 90% sequence identity to any one of these sequences encoding a polypeptide having 2α-hydroxytaxane 2-O-benzoyltransferase activity, or a complementary sequence thereto; a nucleotide sequence encoding a polypeptide having SEQ ID NO: 50, SEQ ID NO: 52, SEQ ID NO: 54, SEQ ID NO: 56, SEQ ID NO: 58, or SEQ ID NO: 60, or at least about 90% sequence identity to any one of these sequences having 2α-hydroxytaxane 2-O-benzoyltransferase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having 2α-hydroxytaxane 2-O-benzoyltransferase activity.

In some embodiments, the moss cell comprises a nucleotide sequence encoding an abietadiene synthase selected from the group consisting of: a nucleotide sequence of SEQ ID NO: 61, SEQ ID NO: 63, SEQ ID NO: 65, or SEQ ID NO: 67, or at least about 90% sequence identity to any one of these sequences encoding a polypeptide having abietadiene synthase activity, or a complementary sequence thereto; a nucleotide sequence encoding a polypeptide having SEQ ID NO: 62, SEQ ID NO: 64, SEQ ID NO: 66, or SEQ ID NO: 68, or at least about 90% sequence identity to any one of these sequences having abietadiene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having abietadiene synthase activity.

In some embodiments, the moss cell comprises a nucleotide sequence encoding an abietadienol/abietadienal oxidase selected from the group consisting of: a nucleotide sequence of SEQ ID NO: 69, or at least about 90% sequence identity to any one of these sequences encoding a polypeptide having abietadienol/abietadienal oxidase activity, or a complementary sequence thereto; a nucleotide sequence encoding a polypeptide having SEQ ID NO: 70, or at least about 90% sequence identity to any one of these sequences having—abietadienol/abietadienal oxidase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having abietadienol/abietadienal oxidase activity.

In some embodiments, the moss cell comprises a nucleotide sequence encoding a ent-copalyl diphosphate synthase selected from the group consisting of: a nucleotide sequence of SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, or SEQ ID NO: 115, or at least about 90% sequence identity to any one of these sequences encoding a polypeptide having ent-copalyl diphosphate synthase activity, or a complementary sequence thereto; a nucleotide sequence encoding a polypeptide having SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, or SEQ ID NO: 116, or at least about 90% sequence identity to any one of these sequences having ent-copalyl diphosphate synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having ent-copalyl diphosphate synthase activity.

In some embodiments, the moss cell comprises a nucleotide sequence encoding an ent-Kaurene synthase selected from the group consisting of: a nucleotide sequence of SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, or SEQ ID NO: 143, or at least about 90% sequence identity to any one of these sequences encoding a polypeptide having ent-Kaurene synthase activity, or a complementary sequence thereto; a nucleotide sequence encoding a polypeptide having SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, or SEQ ID NO: 144, or at least about 90% sequence identity to any one of these sequences having ent-Kaurene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having ent-Kaurene synthase activity.

In some embodiments, the moss cell comprises a nucleotide sequence encoding an ent-Kaurene oxidase selected from the group consisting of: a nucleotide sequence of SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, or SEQ ID NO: 173, or at least about 90% sequence identity to any one of these sequences encoding a polypeptide having ent-Kaurene oxidase activity, or a complementary sequence thereto; a nucleotide sequence encoding a polypeptide having SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, or SEQ ID NO: 174, or at least about 90% sequence identity to any one of these sequences having ent-Kaurene oxidase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having ent-Kaurene oxidase activity.

In some embodiments, the moss cell comprises a nucleotide sequence encoding a kaurenoic acid 13-hydroxylase selected from the group consisting of: a nucleotide sequence of SEQ ID NO: 175, or at least about 90% sequence identity to any one of these sequences encoding a polypeptide having kaurenoic acid 13-hydroxylase activity, or a complementary sequence thereto; a nucleotide sequence encoding a polypeptide having SEQ ID NO: 176, or at least about 90% sequence identity to any one of these sequences having kaurenoic acid 13-hydroxylase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having kaurenoic acid 13-hydroxylase activity.

In some embodiments, the moss cell comprises a nucleotide sequence encoding a UDP-glycosyltransferase UGT85C2 selected from the group consisting of: a nucleotide sequence of SEQ ID NO: 177, or at least about 90% sequence identity to any one of these sequences encoding a polypeptide having UDP-glycosyltransferase activity, or a complementary sequence thereto; a nucleotide sequence encoding a polypeptide having SEQ ID NO: 178, or at least about 90% sequence identity to any one of these sequences having UDP-glycosyltransferase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having UDP-glycosyltransferase activity.

In some embodiments, the moss cell comprises a nucleotide sequence encoding a UDP-glycosyltransferase UGT74G1 selected from the group consisting of: a nucleotide sequence of SEQ ID NO: 179, or at least about 90% sequence identity to any one of these sequences encoding a polypeptide having UDP-glycosyltransferase activity, or a complementary sequence thereto; a nucleotide sequence encoding a polypeptide having SEQ ID NO: 180, or at least about 90% sequence identity to any one of these sequences having UDP-glycosyltransferase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having UDP-glycosyltransferase activity.

In some embodiments, the moss cell comprises a nucleotide sequence encoding a UDP-glycosyltransferase UGT76G1 selected from the group consisting of: a nucleotide sequence of SEQ ID NO: 181, or at least about 90% sequence identity to any one of these sequences encoding a polypeptide having UDP-glycosyltransferase activity, or a complementary sequence thereto; a nucleotide sequence encoding a polypeptide having SEQ ID NO: 182, or at least about 90% sequence identity to any one of these sequences having UDP-glycosyltransferase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having UDP-glycosyltransferase activity.

In some embodiments, the moss cell comprises a nucleotide sequence encoding a CBT-ol cyclase selected from the group consisting of: a nucleotide sequence of SEQ ID NO: 183 or SEQ ID NO: 185, or at least about 90% sequence identity to any one of these sequences encoding a polypeptide having CBT-ol cyclase activity, or a complementary sequence thereto; a nucleotide sequence encoding a polypeptide having SEQ ID NO: 184 or SEQ ID NO: 186, or at least about 90% sequence identity to any one of these sequences having CBT-ol cyclase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having CBT-ol cyclase activity.

In some embodiments, the moss cell comprises a nucleotide sequence encoding a CYP71D16 selected from the group consisting of: a nucleotide sequence of SEQ ID NO: 187, or at least about 90% sequence identity to any one of these sequences encoding a polypeptide having—CYP71D16 activity, or a complementary sequence thereto; a nucleotide sequence encoding a polypeptide having SEQ ID NO: 188, or at least about 90% sequence identity to any one of these sequences having CYP71D16 activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having CYP71D16 activity.

In some embodiments, the moss cell comprises a nucleotide sequence encoding a syn-copalyl diphosphate synthase selected from the group consisting of: a nucleotide sequence of SEQ ID NO: 189, or at least about 90% sequence identity to any one of these sequences encoding a polypeptide having syn-copalyl diphosphate synthase activity, or a complementary sequence thereto; a nucleotide sequence encoding a polypeptide having SEQ ID NO: 190, or at least about 90% sequence identity to any one of these sequences having—syn-copalyldiphosphate synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having syn-copalyl diphosphate synthase activity.

In some embodiments, the moss cell comprises a nucleotide sequence encoding a syn-pimaradiene synthase selected from the group consisting of: a nucleotide sequence of SEQ ID NO: 191 or SEQ ID NO: 193, or at least about 90% sequence identity to any one of these sequences encoding a polypeptide having syn-pimaradiene synthase activity, or a complementary sequence thereto; a nucleotide sequence encoding a polypeptide having SEQ ID NO: 192 or SEQ ID NO: 194, or at least about 90% sequence identity to any one of these sequences having syn-pimaradiene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having syn-pimaradiene synthase activity.

In some embodiments, the moss cell comprises a nucleotide sequence encoding an ent-sandaracopimaradiene synthase selected from the group consisting of: a nucleotide sequence of SEQ ID NO: 195, or at least about 90% sequence identity to any one of these sequences encoding a polypeptide having ent-sandaracopimaradiene synthaseent-sandaracopimaradiene synthase activity, or a complementary sequence thereto; a nucleotide sequence encoding a polypeptide having SEQ ID NO: 196, or at least about 90% sequence identity to any one of these sequences having ent-sandaracopimaradiene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having ent-sandaracopimaradiene synthase activity.

In some embodiments, the moss cell comprises a nucleotide sequence encoding a syn-stemarene synthase selected from the group consisting of: a nucleotide sequence of SEQ ID NO: 197, or at least about 90% sequence identity to any one of these sequences encoding a polypeptide having syn-stemarene synthase activity, or a complementary sequence thereto; a nucleotide sequence encoding a polypeptide having SEQ ID NO: 198, or at least about 90% sequence identity to any one of these sequences having syn-stemarene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having syn-stemarene synthase activity.

In some embodiments, the moss cell comprises a nucleotide sequence encoding an ent-cassadiene synthase selected from the group consisting of: a nucleotide sequence of SEQ ID NO: 199, or at least about 90% sequence identity to any one of these sequences encoding a polypeptide having ent-cassadiene synthase activity, or a complementary sequence thereto; a nucleotide sequence encoding a polypeptide having SEQ ID NO: 200, or at least about 90% sequence identity thereto having ent-cassadiene synthase activity, or a complementary sequence thereto; and an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having ent-cassadiene synthase activity.

In some embodiments, the moss cell comprises a promoter functional in a moss cell; and a transcriptional termination sequence; wherein the promoter, the heterologous nucleic acid molecule, and the transcriptional termination sequence are operably associated in the 5′ to 3′ direction of transcription.

In some embodiments, the moss cell has reduced or eliminated expression or activity for one or more of Mevalonate diphosphate decarboxylase; Mevalonate kinase; HMG-CoA reductase; Squalene epoxidase; 4-Hydroxyphenylpyruvate dioxygenase; Geranylgeranyl pyrophosphate synthase; ent-Kaurene synthetase; Chorismate mutase; Farnesyl pyrophosphate synthase; Phytoene synthase; Adenylate isopentenyltransferase; Squalene-hopene-cyclase; γ-Tocopherol methyltransferase; Geranylgeranyl reductase; Phytoene desaturase; ζ-Carotene desaturase; Geranylgeranyltransferase I; Zeaxanthin epoxidase; Copalyl diphosphate synthase; 2-Heptaprenyl-1,4-naphthoquinone methyltransferase; 9-cis-Epoxycarotenoid cleavage dioxygenase; 1-Deoxy-D-xylulose 5-phosphate synthase; Lycopene ε cyclase; and 2-Methyl-6-phytylhydroquinone 3-methyltransferase.

In some embodiments, the moss cell has reduced or eliminated expression or activity of diterpene synthase or kaurene synthase and the moss cell produces or accumulates decreased levels of ent-kaurene or 16-hydroxykaurane compared to a moss cell not comprising the DNA construct.

Another aspect provides a moss cell comprising at least one terpenoid compound selected from the group consisting of taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin III; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.

Another aspect provides a method of producing a terpenoid compound comprising culturing a moss cell described above. In some embodiments, the method further comprises isolating the terpenoid compound.

Another aspect provides a method of producing a moss cell described above comprising: introducing in the moss cell at least a first heterologous nucleic acid molecule; wherein expression of the heterologous nucleic acid molecule in the cell results in production or accumulation of at least one terpenoid compound selected from the group consisting of taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin III; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of graphs depicting GC-MS analysis of hexane extracts from wild type and transgenic Physcomitrella patens overexpressing taxadiene synthase. FIG. 1A shows GC-MS analysis of hexane extracts from wild type Physcomitrella patens. FIG. 1B shows GC-MS analysis of hexane extracts from transgenic Physcomitrella patens overexpressing taxadiene synthase. A new peak labeled 1 in FIG. 1B is found in transgenic moss but not in wild type. FIG. 1C shows the mass spectral fragmentation pattern of peak 1 of FIG. 1B. The mass spectral fragmentation pattern of peak 1 matches that of taxa-4(5),11(12)-diene. Peaks 2 and 3 have been identified as ent-kaurene and 16-hydroxykaurane, respectively, by comparison of their retention times and mass spectra with authentic chemical standards.

FIG. 2 is a graph depicting GC-MS analysis of hexane extracts from transgenic Physcomitrella patens overexpressing taxadiene synthase (top) and both taxadiene synthase and taxadiene 5-hydroxylase (bottom). Peak 2A is taxadiene-5-ol, based on selected masses (diagnostic of taxadiene-5-ol) used to generate the chromatogram. Note that there is no peak in 1A. The large peak next to 2A is ent-kaurene. Taxadiene was also found in both samples at ˜46.0 min (not shown).

FIG. 3 is a series of graphs depicting GC-MS analysis of hexane extracts from transgenic Physcomitrella patens protonema overexpressing taxadiene synthase and/or taxadiene 5-hydroxylase. FIG. 3A shows transgenic moss protonema overexpressing taxadiene synthase. FIG. 3B shows transgenic moss protonema overexpressing both taxadiene synthase and taxadiene 5-hydroxylase. FIG. 3C shows wild type moss protonema. FIG. 3D shows a mass fragmentation pattern of peak 1A from FIG. 3A. FIG. 3E shows a mass fragmentation pattern of peak 2A from FIG. 3B. FIG. 3F shows a mass fragmentation pattern of peak 3A from FIG. 3B. The unlabelled peak is ent-kaurene.

FIG. 4 is series of graphs depicting GC-MS analysis of hexane extracts from four transgenic P. patens moss gametophyte cultures overexpressing taxadiene synthase and/or taxadiene 5-hydroxylase. All chromatograms (see e.g., FIGS. 4A, 4B, 4C and 4D) are plots of m/z 288 (the molecular weight of oxygenated taxadiene) against retention time. FIG. 4E shows a mass fragmentation pattern of peak 1A from FIG. 4A. FIG. 4F shows a mass fragmentation pattern of peak 2A from FIG. 4B. FIG. 4G shows a mass fragmentation pattern of peak 3A from FIG. 4C. FIG. 4H shows a mass fragmentation pattern of peak 4A from FIG. 4D.

### DETAILED DESCRIPTION OF THE INVENTION

Described herein are compositions and processes for production of terpenoids from metabolically engineered moss. The present invention is based, at least in part, on the discovery that heterologous overexpression of taxadiene synthase in the moss Physcomitrella patens produced terpenoid compounds, such as taxa-4(5),11(12)-diene, without any substantial deleterious phenotypic consequences.

Target terpenoids and corresponding sequences for transformation of a moss cell

According to the approach described herein, a moss host cell can be transformed so as to provide for production or accumulation of a target terpenoid compound. In some embodiments, a target terpenoid compound can be a diterpenoid. In some embodiments, a target terpenoid compound can be a taxoid. A moss host cell can be transformed with a nucleic acid molecule encoding a terpene synthesis enzyme capable of, for example, performing an enzymatic step involved in the metabolism of a target terpenoid. A nucleic acid encoding a terpene synthesis enzyme alone or in any combination can have a substantial effect on the production of the desired terpene compound or in the production of relevant precursors in a transformed moss.

A moss host cell can be transformed so as to accumulate one or more terpenoid compounds. A target terpenoid compound can be endogenous or heterologous. A genetically modified moss can express increased levels of a terpene synthesis enzyme relative to a wild type in the event that the starting moss endogenously comprises a nucleic acid encoding such terpene synthesis enzyme. A genetically modified moss can cause expressed levels of a terpene synthesis enzyme in the event that the starting moss does not endogenously contain a nucleic acid encoding such terpene synthesis enzyme.

For example, a moss host cell can be transformed so as to accumulate one or more of taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin III; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.

One nucleic acid encoding a terpene synthesis enzyme or a combination of several nucleic acid encoding terpene synthesis enzymes can be transformed into a moss host cell, whereby the nucleic acid(s) or enzyme(s) can be modified either in their activity or number in the corresponding host cell. A moss host cell can be further genetically manipulated (e.g., in a key position of a target pathway) such that flux of metabolites can be directed through intermediates useful to the engineered nucleic acid(s) encoding a terpene synthesis enzyme(s). A nucleic acid encoding a terpene synthesis enzyme can be isolated from any suitable organism, e.g. prokaryotes or eukaryotes, which comprises an endogenous sequence recited herein.

A moss host cell can be transformed to express one or more of: taxadiene synthase; taxadiene-5α-hydroxylase; taxadiene-5α-ol-acetyl transferase; taxane-13α-hydroxylase; taxane-10β-hydroxylase; taxoid 14β-hydroxylase; taxoid-9α-hydroxylase; taxoid-2α-hydroxylase; taxoid-7β-hydroxylase; 2α-hydroxytaxane 2-O-benzoyl transferase (i.e., taxoid-2α-O-benzoyl transferase); taxoid C1β-hydroxylase; taxoid C4β, C20-epoxidase; abietadiene synthase; abietadienol/abietadienal oxidase; ent-copalyl diphosphate synthase; ent-Kaurene synthase; ent-Kaurene oxidase; kaurenoic acid 13-hydroxylase; UDP-glycosyltransferase (UGT) converting steviolmonoside to steviolbioside; UGT85C2; UGT74G1; UGT76G1; CBT-ol cyclase; CYP71D16; syn-copalyl diphosphate synthase; syn-pimaradiene synthase; ent-sandaracopimaradiene synthase; syn-stemarene synthase; and ent-cassadiene synthase. Exemplary nucleotide and peptide sequences for such enzymes are provided in Table 1. Designations of terpene synthesis enzymes below refer to GenBank Accession numbers, from which the corresponding sequence for the nucleic acid encoding the terpene synthesis enzyme can be obtained.

TABLE 1
Terpenoid biosynthetic pathway enzymes for transformation of a moss cell

| | GenBank | Nucleotide | Polypeptide |
|---|---|---|---|
| Enzyme | Accession No. | Sequence | sequence |
| taxadiene synthase | U48796 | SEQ ID NO: 1 | SEQ ID NO: 2 |
| | AY007207 | SEQ ID NO: 3 | SEQ ID NO: 4 |
| | AY364469 | SEQ ID NO: 5 | SEQ ID NO: 6 |
| | AY364470 | SEQ ID NO: 7 | SEQ ID NO: 8 |
| | AY365032 | SEQ ID NO: 9 | SEQ ID NO: 10 |
| | DQ305407 | SEQ ID NO: 11 | SEQ ID NO: 12 |
| | AY931015 | SEQ ID NO: 13 | SEQ ID NO: 14 |
| | DQ092389 | SEQ ID NO: 15 | SEQ ID NO: 16 |
| | AY461450 | SEQ ID NO: 17 | SEQ ID NO: 18 |
| taxadiene 5α- | AY289209 | SEQ ID NO: 19 | SEQ ID NO: 20 |
| hydroxylase | AY741375 | SEQ ID NO: 21 | SEQ ID NO: 22 |
| taxadiene-5α-ol- | AY289209 | SEQ ID NO: 23 | SEQ ID NO: 24 |
| acetyl transferase | AY741375 | SEQ ID NO: 25 | SEQ ID NO: 26 |
| | AF190130 | SEQ ID NO: 201 | SEQ ID NO: 202 |
| taxane-13α- | AY056019 | SEQ ID NO: 27 | SEQ ID NO: 28 |
| hydroxylase | AY866412 | SEQ ID NO: 29 | SEQ ID NO: 30 |
| | AY959321 | SEQ ID NO: 31 | SEQ ID NO: 32 |
| taxane-10β- | AF318211 | SEQ ID NO: 33 | SEQ ID NO: 34 |
| hydroxylase | AF545833 | SEQ ID NO: 35 | SEQ ID NO: 36 |
| | AY453403 | SEQ ID NO: 37 | SEQ ID NO: 38 |
| | AY519128 | SEQ ID NO: 39 | SEQ ID NO: 40 |
| taxoid-9α- | | | |
| hydroxylase | | | |
| taxoid-2α- | AY518383 | SEQ ID NO: 41 | SEQ ID NO: 42 |
| hydroxylase | AY789508 | SEQ ID NO: 43 | SEQ ID NO: 44 |
| taxoid-7β- | AY307951 | SEQ ID NO: 45 | SEQ ID NO: 46 |
| hydroxylase | AY374652 | SEQ ID NO: 47 | SEQ ID NO: 48 |
| 2α-hydroxytaxane | AF297618 | SEQ ID NO: 49 | SEQ ID NO: 50 |
| 2-O- | AY675557 | SEQ ID NO: 51 | SEQ ID NO: 52 |
| benzoyltransferase | AY970522 | SEQ ID NO: 53 | SEQ ID NO: 54 |
| | AY972076 | SEQ ID NO: 55 | SEQ ID NO: 56 |
| | AY864799 | SEQ ID NO: 57 | SEQ ID NO: 58 |
| | AY970523 | SEQ ID NO: 59 | SEQ ID NO: 60 |
| taxoid C1β- | | | |
| hydroxylase | | | |
| taxoid C4β, C20- | | | |
| epoxidase | | | |
| abietadiene | U50768 | SEQ ID NO: 61 | SEQ ID NO: 62 |
| synthase | AY473621 | SEQ ID NO: 63 | SEQ ID NO: 64 |
| | AY779541 | SEQ ID NO: 65 | SEQ ID NO: 66 |
| | EU439295 | SEQ ID NO: 67 | SEQ ID NO: 68 |
| Abietadienol/ | AY779537 | SEQ ID NO: 69 | SEQ ID NO: 70 |
| abietadienal | | | |
| oxidase | | | |
| ent-copalyl | U11034 | SEQ ID NO: 71 | SEQ ID NO: 72 |
| diphosphate | AB439590 | SEQ ID NO: 73 | SEQ ID NO: 74 |
| synthase | AB439589 | SEQ ID NO: 75 | SEQ ID NO: 76 |
| | AB439588 | SEQ ID NO: 77 | SEQ ID NO: 78 |
| | XM_002306741 | SEQ ID NO: 79 | SEQ ID NO: 80 |
| | XM_002302074 | SEQ ID NO: 81 | SEQ ID NO: 82 |
| | EU003997 | SEQ ID NO: 83 | SEQ ID NO: 84 |
| | AY242859 | SEQ ID NO: 85 | SEQ ID NO: 86 |
| | AB066271 | SEQ ID NO: 87 | SEQ ID NO: 88 |
| | AB015675 | SEQ ID NO: 89 | SEQ ID NO: 90 |
| | AB169981 | SEQ ID NO: 91 | SEQ ID NO: 92 |
| | AY562491 | SEQ ID NO: 93 | SEQ ID NO: 94 |
| | AY562490 | SEQ ID NO: 95 | SEQ ID NO: 96 |
| | AY602991 | SEQ ID NO: 97 | SEQ ID NO: 98 |
| | AY551435 | SEQ ID NO: 99 | SEQ ID NO: 100 |
| | AB170034 | SEQ ID NO: 101 | SEQ ID NO: 102 |
| | AB109763 | SEQ ID NO: 103 | SEQ ID NO: 104 |
| | AB046689 | SEQ ID NO: 105 | SEQ ID NO: 106 |
| | AB042424 | SEQ ID NO: 107 | SEQ ID NO: 108 |
| | AF049906 | SEQ ID NO: 109 | SEQ ID NO: 110 |
| | AF049905 | SEQ ID NO: 111 | SEQ ID NO: 112 |
| | U63652 | SEQ ID NO: 113 | SEQ ID NO: 114 |
| | AF034545 | SEQ ID NO: 115 | SEQ ID NO: 116 |
| ent-Kaurene | NM_001154587 | SEQ ID NO: 117 | SEQ ID NO: 118 |
| synthase | AY242860 | SEQ ID NO: 119 | SEQ ID NO: 120 |
| | NM_106594 | SEQ ID NO: 121 | SEQ ID NO: 122 |
| | NM_001111787 | SEQ ID NO: 123 | SEQ ID NO: 124 |
| | NM_001111627 | SEQ ID NO: 125 | SEQ ID NO: 126 |
| | E12936 | SEQ ID NO: 127 | SEQ ID NO: 128 |
| | AY347878 | SEQ ID NO: 129 | SEQ ID NO: 130 |
| | AY347877 | SEQ ID NO: 131 | SEQ ID NO: 132 |
| | AB045310 | SEQ ID NO: 133 | SEQ ID NO: 134 |
| | AF097311 | SEQ ID NO: 135 | SEQ ID NO: 136 |
| | AF097310 | SEQ ID NO: 137 | SEQ ID NO: 138 |
| | AF034774 | SEQ ID NO: 139 | SEQ ID NO: 140 |
| | AB003395 | SEQ ID NO: 141 | SEQ ID NO: 142 |
| | U43904 | SEQ ID NO: 143 | SEQ ID NO: 144 |
| ent-Kaurene | NM_122491 | SEQ ID NO: 145 | SEQ ID NO: 146 |
| oxidase | DQ200952 | SEQ ID NO: 147 | SEQ ID NO: 148 |
| | AY995178 | SEQ ID NO: 149 | SEQ ID NO: 150 |
| | AY364317 | SEQ ID NO: 151 | SEQ ID NO: 152 |
| | AY660666 | SEQ ID NO: 153 | SEQ ID NO: 154 |
| | AY660665 | SEQ ID NO: 155 | SEQ ID NO: 156 |
| | AY660664 | SEQ ID NO: 157 | SEQ ID NO: 158 |
| | AY579214 | SEQ ID NO: 159 | SEQ ID NO: 160 |
| | AY462247 | SEQ ID NO: 161 | SEQ ID NO: 162 |
| | AY563549 | SEQ ID NO: 163 | SEQ ID NO: 164 |
| | AY245442 | SEQ ID NO: 165 | SEQ ID NO: 166 |
| | AF212990 | SEQ ID NO: 167 | SEQ ID NO: 168 |
| | AF047721 | SEQ ID NO: 169 | SEQ ID NO: 170 |
| | AF047720 | SEQ ID NO: 171 | SEQ ID NO: 172 |
| | AF047719 | SEQ ID NO: 173 | SEQ ID NO: 174 |
| Kaurenoic acid 13- | Reg No | SEQ ID NO: 175 | SEQ ID NO: 176 |
| hydroxylase | 181186-97-0 | | |
| UDP- | AY345978 | SEQ ID NO: 177 | SEQ ID NO: 178 |
| glycosyltransferase | | | |
| UGT85C2 | | | |
| UGT74G1 | AY345982 | SEQ ID NO: 179 | SEQ ID NO: 180 |
| UGT76G1 | AY345974 | SEQ ID NO: 181 | SEQ ID NO: 182 |
| CBT-ol cyclase | AY049090 | SEQ ID NO: 183 | SEQ ID NO: 184 |
| | AF401234 | SEQ ID NO: 185 | SEQ ID NO: 186 |
| CYP71D16 | AF166332 | SEQ ID NO: 187 | SEQ ID NO: 188 |
| syn-copalyl | AB066270 | SEQ ID NO: 189 | SEQ ID NO: 190 |
| diphosphate | | | |
| synthase | | | |
| syn-pimaradiene | AY616862 | SEQ ID NO: 191 | SEQ ID NO: 192 |
| synthase | AB126934 | SEQ ID NO: 193 | SEQ ID NO: 194 |
| ent- | DQ823355 | SEQ ID NO: 195 | SEQ ID NO: 196 |
| sandaracopimaradiene | | | |
| synthase | | | |
| syn-stemarene | AB118056 | SEQ ID NO: 197 | SEQ ID NO: 198 |
| synthase | | | |
| ent-cassadiene | DQ823354 | SEQ ID NO: 199 | SEQ ID NO: 200 |
| synthase | | | |

A moss host cell can be transformed with a heterologous nucleotide sequence recited in Table 1, or with a nucleotide sequence having at least about 90% sequence identity to a heterologous nucleotide sequence recited in Table 1 that encodes a polypeptide having a specified enzymatic activity, or a complementary sequence to any of these sequences. For example, a moss host cell can be transformed with a nucleotide sequence having at least about 91%, at least about 92%, at least about 93%, at least about 94%, at least about 95%, at least about 96%, at least about 97%, at least about 98%, or at least about 99% sequence identity to a heterologous nucleotide sequence recited in Table 1 that encodes a polypeptide having a specified enzymatic activity corresponding to that heterologous nucleotide sequence.

A moss host cell can be transformed with a heterologous nucleotide sequence encoding a polypeptide sequence recited in Table 1, or encoding a polypeptide sequence having at least about 90% sequence identity to a polypeptide sequence recited in Table 1 having a specified enzymatic activity, or a complementary sequence to any of these sequences. For example, a moss host cell can be transformed with a heterologous nucleotide sequence encoding a polypeptide sequence having at least about 91%, at least about 92%, at least about 93%, at least about 94%, at least about 95%, at least about 96%, at least about 97%, at least about 98%, or at least about 99% sequence identity to a polypeptide sequence recited in Table 1 that retains a corresponding enzymatic activity.

A moss host cell can be transformed with an isolated polynucleotide that hybridizes to any of the above discussed nucleic acid sequences under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having a specified enzymatic activity corresponding to the above discussed nucleic acid sequence.

A terpenoid compound synthesized in a moss host cell can be accumulated, converted to another terpenoid compound, or both. Generally, a terpenoid compound will have greater levels of accumulation where no additional terpenoid biosynthetic enzyme with specificity for that terpenoid compound has been engineered into the moss host cell. In some embodiments, a terpenoid compound produced from a first heterologous enzyme will accumulate in the moss cell. In some embodiments, a terpenoid compound produced from a first heterologous enzyme, where the host cell has at least a second heterologous enzyme expressing a second terpenoid biosynthetic enzyme with at least some specificity for the terpenoid compound, will accumulate in the moss cell. In some embodiments, a terpenoid compound produced from a first heterologous enzyme, where the host cell has at least a second heterologous enzyme expressing a second terpenoid biosynthetic enzyme with at least some specificity for the terpenoid compound, will be produced in the moss cell but will not substantially accumulate in the moss cell.

To produce taxa-4(5),11(12)-diene, a precursor of the anticancer drug paclitaxel, a moss host cell can be engineered to express taxadiene synthase. As shown herein, overexpression of taxadiene synthase (from Taxus brevifolia) in P. patens can result in production or accumulation of taxa-4(5),11(12)-diene (see Example 1).

To produce a taxadien-ol, a moss host cell can be engineered to express taxadiene synthase and one or more hydroxylases. As shown herein, overexpression of taxadiene synthase and taxadiene 5-hydroxylase in P. patens can result in production or accumulation of a taxadien-ol (see Examples 2-3). For example, accumulated taxadien-ol can include one or more of taxadiene-5-ol, taxadien-11-ol, taxadien-18-ol, and taxadien-20-ol. For example, a moss host cell can be engineered to express taxadiene synthase and a one or more hydroxylases selected from taxadiene 5-hydroxylase; taxane-13α-hydroxylase; taxane-10β-hydroxylase; taxoid 14β-hydroxylase; taxoid-9α-hydroxylase; taxoid-2α-hydroxylase; taxoid-7β-hydroxylase; taxoid C1β-hydroxylase. In some embodiments, a moss host cell can be engineered to express taxadiene synthase and taxadiene 5-hydroxylase along with one or more additional hydroxylases, such as taxane-13α-hydroxylase; taxane-10β-hydroxylase; taxoid 14β-hydroxylase; taxoid-9α-hydroxylase; taxoid-2α-hydroxylase; taxoid-7β-hydroxylase; and taxoid C1β-hydroxylase. In some of these embodiments, the most host cell can accumulate, for example, 5α-acetoxy-taxadiene; 5α-acetoxy-10β-hydroxy-taxadiene; 5α-acetoxy-10β,14β-dihydroxy-taxadiene; or 5α,13α-dihydroxy-taxadiene.

In some embodiments, a moss host cell described above is further engineered to express additional taxoid biosynthetic enzymes. For example, a moss host cell can be engineered to express taxadiene synthase, one or more hydroxylases, and one or more transferases. In some embodiments, a moss host cell is can be engineered to express taxadiene synthase, a hydroxylase (e.g., taxadiene 5-hydroxylase), and an acetyl transferase (e.g., taxadiene-5α-ol-acetyl transferase).

To produce taxadiene-5-ol, a moss host cell can be engineered to express taxadiene synthase and taxadiene 5α-hydroxylase. As shown herein, overexpression of taxadiene synthase and taxadiene 5-hydroxylase in P. patens can result in production or accumulation of taxadiene and taxadiene-5-ol (see Example 2).

To produce taxadien-11-ol, a moss host cell can be engineered to express taxadiene synthase and taxadiene 5α-hydroxylase. As shown herein, overexpression of taxadiene synthase and taxadiene 5-hydroxylase in P. patens can result in production or accumulation of taxadien-11-ol (see Example 3).

To produce taxadien-18-ol, a moss host cell can be engineered to express taxadiene synthase and taxadiene 5α-hydroxylase. As shown herein, overexpression of taxadiene synthase and taxadiene 5-hydroxylase in P. patens can result in production or accumulation of taxadien-18-ol (see Example 3).

To produce taxadien-20-ol, a moss host cell can be engineered to express taxadiene synthase and taxadiene 5α-hydroxylase. As shown herein, overexpression of taxadiene synthase and taxadiene 5-hydroxylase in P. patens can result in production or accumulation of taxadien-20-ol (see Example 3).

To produce 5(12)-oxa-3(11)-cyclotaxane, a moss host cell can be engineered to express taxadiene synthase and taxadiene 5α-hydroxylase. As shown herein, overexpression of taxadiene synthase and taxadiene 5-hydroxylase in P. patens can result in production or accumulation of 5(12)-oxa-3(11)-cyclotaxane (see Example 3).

To produce taxadiene-5α-acetoxy-13β-ol, a moss host cell can be engineered to express taxadiene synthase; taxadiene-5α-hydroxylase; taxadiene-5α-ol-acetyl transferase; and taxane-13α-hydroxylase.

To produce taxadiene-5α,13α-diol, a moss host cell can be engineered to express taxadiene synthase; taxadiene-5α-hydroxylase; and taxane-13α-hydroxylase.

To produce taxadiene-5α-acetoxy-10β-ol, a moss host cell can be engineered to express taxadiene synthase; taxadiene-5α-hydroxylase; taxadiene-5α-ol-acetyl transferase; and taxane-10β-hydroxylase.

To produce 10-deacetylbaccatin III, a moss host cell can be engineered to express taxadiene synthase; taxadiene-5α-hydroxylase; taxadiene-5α-ol-acetyl transferase; taxane-10β-hydroxylase; taxane-13α-hydroxylase; taxoid-9α-hydroxylase; taxoid-2α-hydroxylase; taxoid-7β-hydroxylase; 2α-hydroxytaxane 2-O-benzoyltransferase (i.e., taxoid-2α-O-benzoyl transferase); taxoid C1β-hydroxylase; and taxoid C4β, C20-epoxidase.

To produce abietadiene, a moss host cell can be engineered to express abietadiene synthase.

To produce abietic acid, a moss host cell can be engineered to express abietadiene synthase and abietadienol/abietadienal oxidase.

To produce steviol, a moss host cell can be engineered to express ent-copalyl diphosphate synthase; ent-Kaurene synthase; ent-Kaurene oxidase; and kaurenoic acid 13-hydroxylase.

To produce steviolmonoside, a moss host cell can be engineered to express ent-copalyl diphosphate synthase; ent-Kaurene synthase; ent-Kaurene oxidase; kaurenoic acid 13-hydroxylase; and UDP-glycosyltransferase (UGT) UGT85C2 converting steviolmonoside to steviolbioside.

To produce stevioside, a moss host cell can be engineered to express ent-copalyl diphosphate synthase; ent-Kaurene synthase; ent-Kaurene oxidase; kaurenoic acid 13-hydroxylase; UDP-glycosyltransferase (UGT) UGT85C2 converting steviolmonoside to steviolbioside; and UGT74G1.

To produce rebaudioside A, a moss host cell can be engineered to express ent-Copalyl diphosphate synthase; ent-Kaurene synthase; ent-Kaurene oxidase; kaurenoic acid 13-hydroxylase; UDP-glycosyltransferase (UGT) UGT85C2 converting steviolmonoside to steviolbioside; UGT74G1; and UGT76G1.

To produce kaurenoic acid, a moss host cell can be engineered to express ent-Copalyl diphosphate synthase; ent-Kaurene synthase; and ent-Kaurene oxidase.

To produce cembranoids, a moss host cell can be engineered to express CBT-ol cyclase and CYP71D16.

To produce momilactones A and B, a moss host cell can be engineered to express syn-copalyl diphosphate synthase and syn-pimaradiene synthase.

To produce oryzalexins A-F, a moss host cell can be engineered to express ent-Copalyl diphosphate synthase and ent-sandaracopimaradiene synthase.

To produce oryzalexin S, a moss host cell can be engineered to express syn-copalyl diphosphate synthase and syn-stemarene synthase.

To produce phytocassanes A-E, a moss host cell can be engineered to express ent-Copalyl diphosphate synthase and ent-cassadiene synthase.

Down-Regulated Endogenous Moss Genes

A moss host cell can be transformed so as to reduce an endogenous terpenoid compound. Reduction of an endogenous terpenoid compound can increase precursor availability for engineered pathways associated with production of a target terpenoid. Reduction of an endogenous terpenoid compound can increase production or accumulation of a target terpenoid compound in a moss host cell.

A moss host cell can be transformed so as to reduce or eliminate one or more of Mevalonate diphosphate decarboxylase; Mevalonate kinase; HMG-CoA reductase; Squalene epoxidase; 4-Hydroxyphenylpyruvate dioxygenase; Geranylgeranyl pyrophosphate synthase; ent-Kaurene synthetase; Chorismate mutase; Farnesyl pyrophosphate synthase; Phytoene synthase; Adenylate isopentenyltransferase; Squalene-hopene-cyclase; γ-Tocopherol methyltransferase; Geranylgeranyl reductase; Phytoene desaturase; ζ-Carotene desaturase; Geranylgeranyltransferase I; Zeaxanthin epoxidase; Copalyl diphosphate synthase; 2-Heptaprenyl-1,4-naphthoquinone methyltransferase; 9-cis-Epoxycarotenoid cleavage dioxygenase; 1-Deoxy-D-xylulose 5-phosphate synthase; Lycopene ε cyclase; and 2-Methyl-6-phytylhydroquinone 3-methyltransferase.

For example, moss genes encoding proteins involved in the synthesis of tocopherols and carotenoids, along with antisense nucleic acid molecules specific thereto, are described in US App Pub No. 2003/0157592. Expression of such endogenous gene targets can be reduced or eliminated, for example by the recited antisense molecules, so as to increase precursor availability or increase production or accumulation of a target terpenoid compound in a moss host cell.

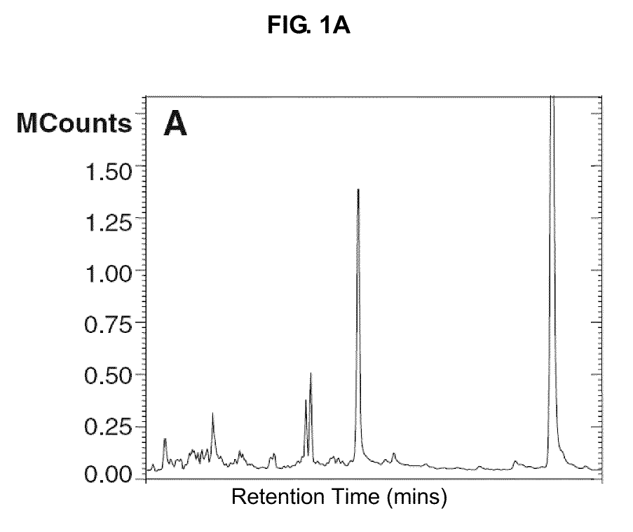

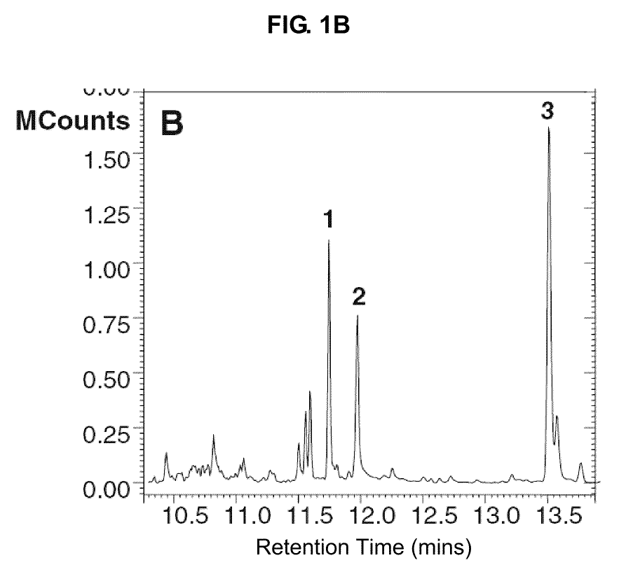

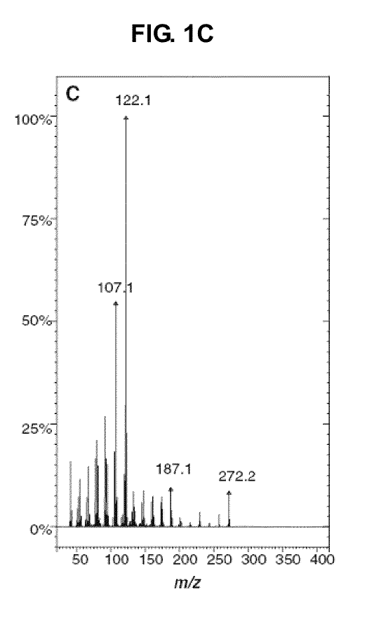

P. patens has been reported to produce two diterpenoids (ent-kaurene and 16-hydroxykaurane) as secondary metabolites (von Schwartzenberg et al. 2004), which together can comprise up to 0.2% of its fresh weight (based on GC-MS analysis). As shown herein, P. patens overexpressing taxadiene synthase or taxadiene synthase and taxadiene 5-hydroxylase continue to produce ent-kaurene and 16-hydroxykaurane as a major portion of the diterpenoid pool (see e.g., FIG. 1; Examples 1-2). Because P. patens produces high levels (up to 0.2% fresh weight) of ent-kaurene and 16-hydroxykaurane, the amounts of a target terpenoid compound can be increased by inhibiting formation of ent-kaurene or 16-hydroxykaurane.

Terpenoid production in a transgenic moss overexpressing a heterologous terpene synthesis gene can produce elevated levels of a target terpenoid by eliminating or reducing an endogenous terpene synthesis gene(s), such as ent-kaurene or 16-hydroxykaurane formation. For example, knocking out the moss bifunctional diterpene synthase gene can reduce or eliminate levels of endogenous ent-kaurene or 16-hydroxykaurane. In some embodiments, diterpene synthase activity can be reduced or eliminated to increase production or accumulation of other target terpenoids. Knocking out the moss kaurene synthase gene can reduce or eliminate levels of endogenous ent-kaurene or 16-hydroxykaurane. In some embodiments, kaurene synthase activity can be reduced or eliminated to increase production or accumulation of other target terpenoids. Knocking out moss terpenoid synthesis genes can be accomplished by, for example, homologous recombination or RNAi (see e.g., Schaefer 2002 Annu Rev Plant Biol. 53, 477-501; Bezanilla et al. 2003 Plant Physiol. 133(2), 470-4).

Promoters

A terpene synthesis gene can be operably linked to a promoter for transformation of a plant cell. The promoter can be any promoter functional in a moss cell (see e.g., Weise et al. Applied Microbiology and Biotechnology 70(3), 337-345; Saidi et al. 2005 Plant Molecular Biology 59(5), 697-711; Horstmann et al. 2004 BMC Biotechnology 4; Holtorf et al. 2002 Plant Cell Reports 21(4), 341-346; Zeidler et al. 1996 Plant Molecular Biology 30(1), 199-205). The promoter can be an inducible promoter.

Examples of promoters than can be used in accord with methods and compositions described herein include, but are not limited to, ubiquitin promoter (see e.g., Example 1); factor EF1α gene promoter (US App Pub No. 2008/0313776); rice tungro bacilliform virus (RTBV) gene promoter (US App Pub No. 2008/0282431); cestrum yellow leaf curling virus (CmYLCV) promoter (Stavolone et al. Plant Molecular Biology 53(5), 663-673); tCUP cryptic promoter system (Malik et al. 2002 Theoretical and Applied Genetics 105(4), 505-514); T6P-3 promoter (JP2002238564); S-adenosyl-L-methionine synthetase promoter (WO/2000/037662); Raspberry E4 gene promoter (U.S. Pat. No 6,054,635); cauliflower mosaic virus 35S promoter (Benfey et al. 1990 Science 250(4983), 959-966); figwort mosaic virus promoter (U.S. Pat. No. 5,378,619); conditional heat-shock promoter (Saidi et al. 2005 Plant Molecular Biology 59(5), 697-711); promoter subfragments of the sugar beet V-type H+-ATPase subunit c isoform (Holtorf et al. 2002 Plant Cell Reports 21(4), 341-346); beta-tubulin promoter (Jost et al. 2005 Current Genetics 47(2), 111-120); and bacterial quorum-sensing components (You et al. 2006 Plant Physiology 140 (4), 1205-1212).

Moss Host Cells

Overexpression of a terpene synthesis gene in a moss can avoid problems in post-translational modification of heterologously expressed plant enzymes and formation of multi-enzyme complexes because it is a plant cell. Yeasts and bacteria do not have the same post-translational modification mechanisms as plants, which may be required by plant enzymes for optimal activity. As such, yeast and bacteria host cells may not properly utilize metabolons (i.e., multienzyme complexes) for coupling consecutive steps in a pathway, which could explain why individual expression of taxadiene synthase (Huang et al. 2001), taxadiene-5α-hydroxylase (Hefner et al. 1996), taxadiene-5α-ol acetyltransferase (Walker et al. 2000), taxane 10β-hydroxylase (Schoendorf et al. 2001), and taxane 13α-hydroxylase (Jennewein et al. 2001) in yeast or bacteria resulted in active enzymes, yet simultaneous expression of these genes in yeast did not result in the production of the expected downstream products, except for taxadiene 5α-ol (DeJong et al. 2006).

Overexpression of a terpene synthesis gene in a moss can avoid problems of stunted growth phenotype observed with overexpression of such genes in higher plants. As demonstrated herein, there were no phenotypic differences observed between transgenic P. patens and the wild type (see e.g., Examples 1-2). In contrast, both arabidopsis and tomato had reduced growth when they overexpressed taxadiene synthase (Besumbes et al. 2004; Kovacs et al. 2007). Presumably, the introduced taxadiene pathway in arabidopsis and tomato interfered with endogenous gibberellin biosynthesis (Besumbes et al. 2004), which used the same precursor (geranylgeranyl diphosphate) as taxa-4(5),11(12)-diene. Mosses do not require gibberellins for growth (Yasumura et al. 2007; Hirano et al. 2007; Vandenbussche et al. 2007), which may account for why growth was not inhibited in transgenic P. patens.

In some embodiments, the moss host belongs to the genus Physcomitrella or Ceratodon. In one embodiment, the moss host belongs to the genus Physcomitrella. An example of a Physcomitrella moss that can be transformed to accumulate a terpenoid includes, but is not limited to, Physcomitrella patens. An example of a Ceratodon moss that can be transformed to accumulate a terpenoid includes, but is not limited to, Ceratodon purpureus.

Moss host cells can be transformed according to molecular methods understood in the art, as discussed further below. For example, moss transformation protocols are described in Nogue et al. 2007 Research & Reviews in BioSciences 1(1), 27-34; Weise et al. Applied Microbiology and Biotechnology 70(3), 337-345; Saidi et al. 2005 Plant Molecular Biology 59(5), 697-711; and US App Pub No. 2003/0157592.

Processes for culture of moss are known in the art (see e.g., Decker and Reski 2008; Knight et al. 2002 Molecular Plant Biology 2, 285-301; Cove et al. 2009 Emerging Model Organisms: A Laboratory Manual, Vol. 1. CSHL Press, Cold Spring Harbor, N.Y., USA, 2009). Except as otherwise noted herein, therefore, the culturing of a transgenic moss described herein can be carried out in accordance with such processes.

Molecular Engineering

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Gilmartin and Bowler (2002) Molecular Plant Biology Volume 1, Oxford University Press, ISBN-10: 0199638756; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, Agrobacterium-mediated transformation, direct DNA uptake by protoplasts, and the like. The transfected cells can be selected and propagated to provide recombinant host cells that comprise the expression vector stably integrated in the host cell genome.

For example, moss transformation protocols are described in Nogue et al. 2007 Research & Reviews in BioSciences 1(1), 27-34; Weise et al. Applied Microbiology and Biotechnology 70(3), 337-345; Saidi et al. 2005 Plant Molecular Biology 59(5), 697-711; and US App Pub No. 2003/0157592.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Exemplary nucleic acids which may be introduced to a moss host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell, e.g. mosses (back-translated nucleic acid sequences).

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Method

Another aspect provides a method for producing a target terpenoid. Such method can involve either the culturing of a transformed moss cell, tissue, organ, or culturing a whole moss described herein, such that a target terpenoid is produced. The method can involve transforming a moss host cell as described above. The method can include the step of recovering the target terpenoid from the cultured moss. Suitable protocols for identifying, isolating, or purifying a target terpenoid from the cultured moss are generally understood in the art.

In some embodiments, methods described herein include induction of taxoid synthesis exposure or contact of the transgenic moss (e.g., cell, tissue, organ, or whole moss) with an agent that stimulates synthesis of one or more target compounds. For example, a transgenic moss as described herein can be contacted or exposed to methyl jasmonate so as to induce taxoid production.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims.

Furthermore, it should be appreciated that all examples in the present disclosure are provided as non-limiting examples.

### Examples

The following non-limiting examples are provided to further illustrate the present invention. It should be appreciated by those of skill in the art that the techniques disclosed in the examples that follow represent approaches the inventors have found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

This example describes transformation of Physcomitrella patens with heterologous taxadiene synthase resulting in production or accumulation of taxadiene in the cultured transformed moss.

The coding region of taxadiene synthase from Taxus brevifolia was amplified by PCR using oligonucleotides 5′-CACCATGGCTCAGCTCTCATTTAAT-3′ (SEQ ID NO: 203) and 5′-TCATACTTGAATTGGATCAATATAAACTTT-3′ (SEQ ID NO: 204) as primers, and taxadiene synthase cDNA (SEQ ID NO: 1) as template. The amplified product (about 2.6 kb) was gel purified and cloned into a pENTR vector (Invitrogen) via a Topoisomerase-mediated ligation reaction, and then subcloned into an expression vector (pTHUBlgateway, SEQ ID NO: 213, constructed from pGEMT-easy of Promega) via a Gateway LR reaction using LR clonase II, to generate the plasmid pTHUBI:TS. This plasmid placed taxadiene synthase under the control of a ubiquitin promoter.

After verification of the inserted taxadiene synthase gene in pTHUBI:TS by DNA sequencing, pTHUBI:TS was linearized using the restriction enzyme SwaI and then transformed into Physcomitrella patens (Gransden strain) protoplasts using standard polyethylene glycol-mediated transformation procedure (Schaefer and Zryd 1997). Hygromycin resistant P. patens transformants were isolated and grown according to Perroud and Quatrano (2006). After screening transformants for targeted gene insertions by PCR, two transgenic lines (TS3 and TS9) showed the presence of the transgene at the targeted locus. Southern blot analysis performed independently with hygromycin and taxadiene synthase probes confirmed the presence of multicopy insertion of the vector in both lines. Presence of taxadiene synthase protein (about 75 kDa) (see SEQ ID NO: 2) was verified by western blot (data not shown) using rabbit polyclonal antibody raised against taxadiene synthase. TS3 and TS9 lines were then further analyzed, along with a wild type control, for the presence of taxa-4(5),11(12)-diene, as described below.

Hexane (1 ml) was added to about 100 mg (fresh wt) transgenic or wild type P. patens tissues, which were then homogenized in the presence of 1 g Zirconia beads (Fisher) by shaking on a FastPrep instrument (setting 6.0, 3 9 20 s). After centrifugation at 16,000 g for 2 min, the hexane supernatant (0.5 ml) was transferred into a glass vial, from which 5 μl was injected (in splitless mode) by a Varian CP-8410 autoinjector into a FactorFour™ 5 ms capillary column that was installed on a Varian 3900 gas chromatograph connected to a Saturn 2100T ion trap mass spectrometer. Using a temperature program that runs from 50° C. (initially held for 1 min) to 250° C. at a rate of 20° C. per minute, a unique peak at 11.75 min (see e.g., Peak 1, FIG. 1B) was observed in extracts from both TS3 (data not shown) and TS9 (see e.g., FIG. 1B). The mass fragmentation pattern of this peak (see e.g., FIG. 1C) had the same diagnostic ions (m/z 107, 121, 122, 123, 229, 257 and 272) as that reported for taxa-4(5),11(12)-diene (Wildung and Croteau 1996). The amounts of ent-kaurene (see e.g., Peak 2, FIG. 1B) and 16-hydroxykaurane (see e.g., Peak 3, FIG. 1B) in the samples have been quantified by comparison of peak areas with authentic standards.

In the absence of taxa-4(5),11(12)-diene standard, an identical retention time could not be verified for this peak with taxa-4(5),11(12)-diene. Nevertheless, the presence of a taxadiene synthase gene and protein in the transgenic moss (confirmed by Southern and western blots, respectively) and the de novo formation of a compound with the same fragmentation pattern as taxa-4(5),11(12)-diene in transgenic mosses (but not in the wild type) (see e.g., FIG. 1A) constitute sufficient evidence to suggest that P. patens has for the first time been metabolically engineered to produce taxa-4(5),11(12)-diene. Using nonadecane as an internal standard, it was estimated that taxa-4(5),11(12)-diene could be produced by P. patens up to 0.05% of its fresh weight (maximum value among 12 independent extractions of TS3 and TS9).

GC-MS analysis of hexane extracts from transgenic Physcomitrella patens overexpressing taxadiene synthase showed a new peak found in transgenic moss but not in wild type (see e.g., peak 1 in FIG. 1B). The mass spectral fragmentation pattern of peak 1 (see e.g., FIG. 1C) matches that of taxa-4(5),11(12)-diene. Peaks 2 and 3 have been identified as ent-kaurene and 16-hydroxykaurane, respectively, by comparison of their retention times and mass spectra with authentic chemical standards (data not shown).

Results showed that, in stable moss transformants, taxa-4(5),11(12)-diene was produced up to 0.05% fresh weight of tissue, without significantly affecting the amounts of the endogenous diterpenoids (ent-kaurene and 16-hydroxykaurane). Unlike higher plants that had been genetically modified to produce taxa-4(5),11(12)-diene, transgenic P. patens did not exhibit growth inhibition due to alteration of diterpenoid metabolic pools.

Results also demonstrated there were no phenotypic differences observed between transgenic P. patens and the wild type when examined under 10× to 40× magnification (data not shown).

### Example 2

This example describes transformation of Physcomitrella patens with heterologous taxadiene synthase and taxadiene 5-hydroxylase, resulting in production or accumulation of taxadiene and taxadiene-5-ol in the cultured transformed moss.

Methods are as described in Example 1, unless otherwise indicated. Taxadiene synthase was overexpressed under the control of a ubiquitin promoter. Taxadiene 5-hydroxylase was overexpressed under the control of a 35S promoter.

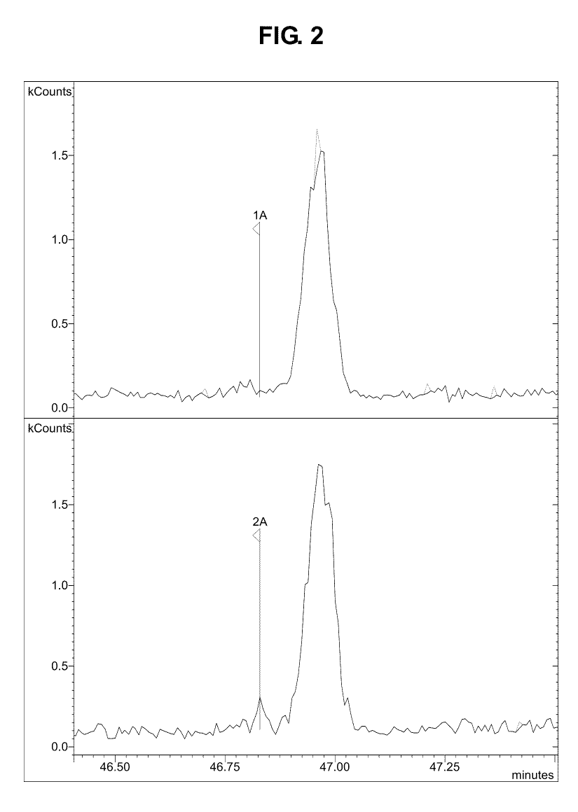

The gene taxadiene-5α-hydroxylase (T5H) (SEQ ID NO: 19) was amplified by PCR, cloned into pENTR, and then the pENTR/T5H was transformed into TOP10 E. coli. The pENTR/T5H plasmid was equipped with an antibiotic marker (ampicillin), which was used for screening analysis. Once a suitable colony was identified, it was grown overnight. At this point, the bacteria were concentrated by centrifugation and the plasmid recovered. The T5H gene was then identified using gel electrophoresis and PCR analysis. From here, the T5H gene was transferred to a destination vector pTZ35Sgateway, using the Gateway system, and the resulting pTZ35Sgateway/T5H plasmid was transformed into OmniMaxE. coli. The colonies were again screened using kanamycin and PCR, and positive colonies were grown overnight. After the bacteria were collected by centrifugation, the plasmid was isolated and analyzed using gel electrophoresis and PCR. The plasmid was then transformed into the moss P. patens expressing the taxadiene synthase (TS) gene, and the presence of taxadiene and taxadiene-ol was verified using GC-MS (see e.g., FIG. 2).

The transgenic moss which has incorporated both the taxadiene synthase (TS) and taxadiene-5α-hydroxylase (T5H) was compared to the moss which only incorporated taxadiene synthase. The concentration of taxadiene in the moss containing both TS and T5H is less than that found in the moss expressing only TS. This result is consistent with the conversion of taxadiene into taxadiene-ol. The results also indicate that taxadiene-ol is present in the moss that contains both T5H and TS, but not in the moss that only contains TS.

Results showed that taxadiene was produced up to 0.05% fresh weight of moss tissue, while trace amounts of taxadiene-ol were detected by GC-MS (see e.g., FIG. 2, peak 2A). The amounts of endogenous diterpenoids (ent-kaurene and 16-hydroxykaurane) in the transformed moss were not significantly affected. Transgenic P. patens did not exhibit any detrimental phenotypes, unlike higher plants that had been genetically modified to produce taxadiene.

The above experiments represent the first time that taxadiene-ol has been produced in any plant.

### Example 3

This example describes analysis of accumulated taxoids in transgenic P. patents expressing heterologous taxadiene synthase and taxadiene 5-hydroxylase.

Methods are as described in Examples 1-2, unless otherwise indicated.

An overexpression plasmid harboring the taxadiene-5α-hydroxylase gene (SEQ ID NO: 19) was transformed into the moss P. patens, which already carried the taxadiene synthase gene (SEQ ID NO: 1) from a previous transformation, as described in Example 2. Both genes were stably integrated into the genome and under the control of constitutive promoters (ubiquitin and 35S respectively).

Stable transformants of P. patens that overexpressed both taxadiene synthase and taxadiene-5α-hydroxylase were analyzed by gas chromatography and mass spectrometry.

Results showed transgenic mosses produced three taxoids, identified as taxadien-5-ol (as described in Example 2) along with 5(12)-oxa-3(11)-cyclotaxane; taxadien-11-ol; taxadien-18-ol; and taxadien-20-ol.

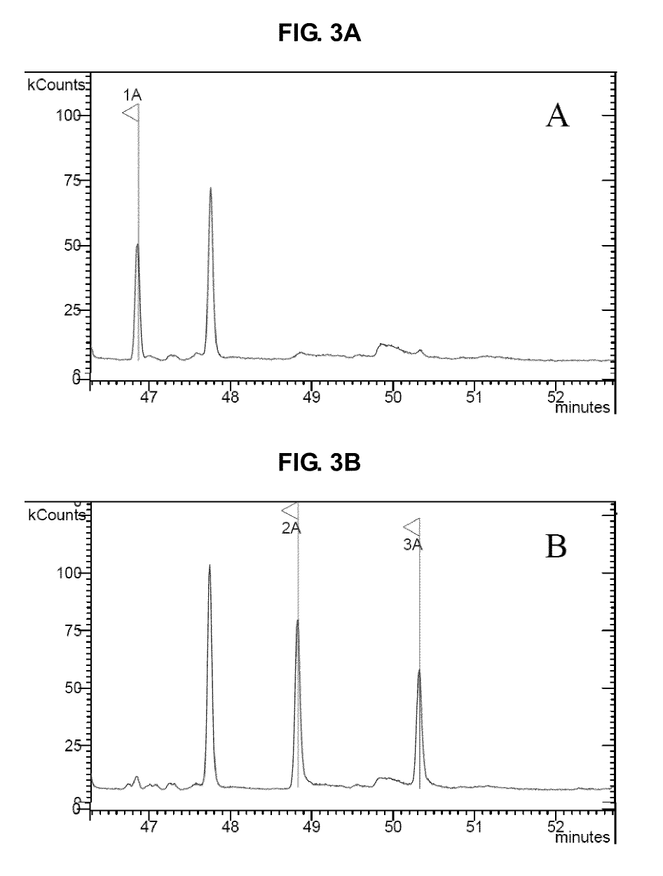

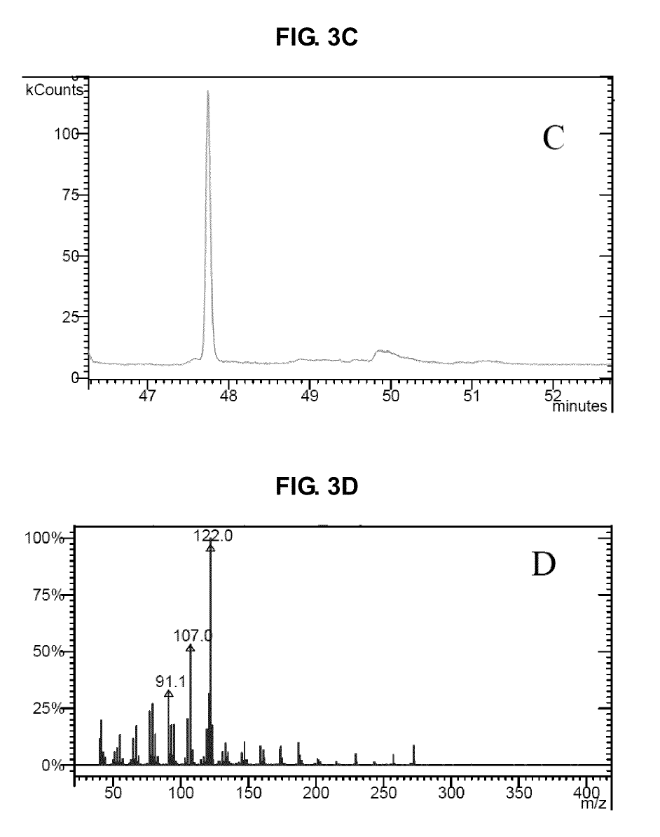

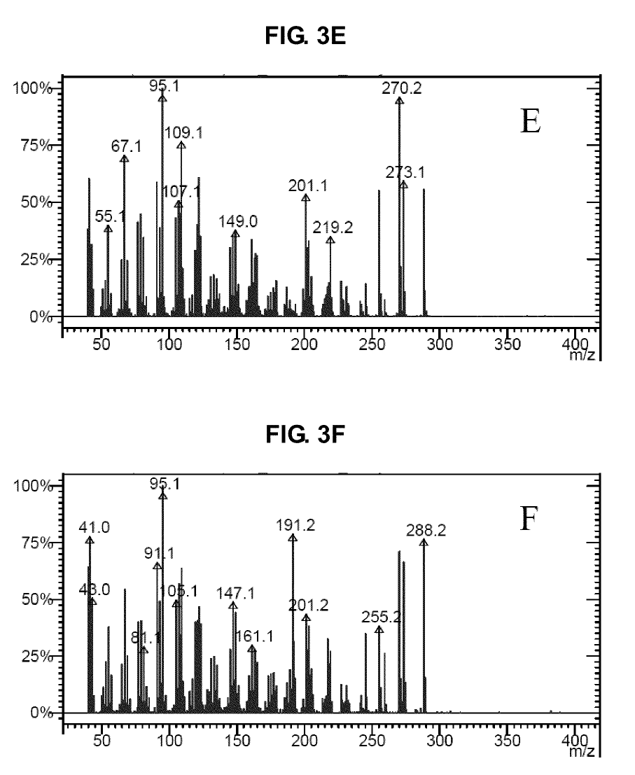

GC-MS analysis of terpenes was performed in transgenic moss protonema overexpressing taxadiene synthase (see e.g., FIG. 3A), transgenic moss protonema overexpressing both taxadiene synthase and taxadiene 5-hydroxylase (see e.g., FIG. 3B), and wild type moss protonema (see e.g., FIG. 3C). Overexpression of taxadiene synthase resulted in the formation of a peak (see e.g., peak 1A of FIG. 3A), which is not present in the wild type (see e.g., FIG. 3C). Peak 1A of FIG. 3A had a mass fragmentation pattern consistent with it being taxadiene (see e.g., FIG. 3D). Overexpression of taxadiene 5-hydroxylase together with taxadiene synthase resulted in a decrease in the above referenced peak 1A and the appearance of two other peaks (see e.g., peaks 2A and 3A in FIG. 3B). Peak 2A of FIG. 3B had a mass fragmentation pattern consistent with it being taxadien-20-ol (see FIG. 3E), while peak 3B of FIG. 3B had a mass fragmentation pattern of 5(12)-oxa-3(11)-cyclotaxane (see e.g., FIG. 3F).

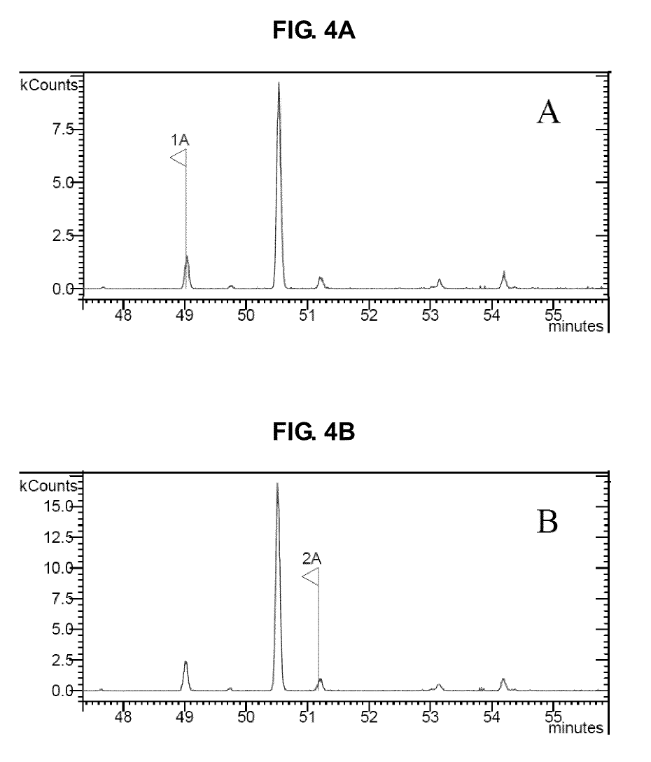

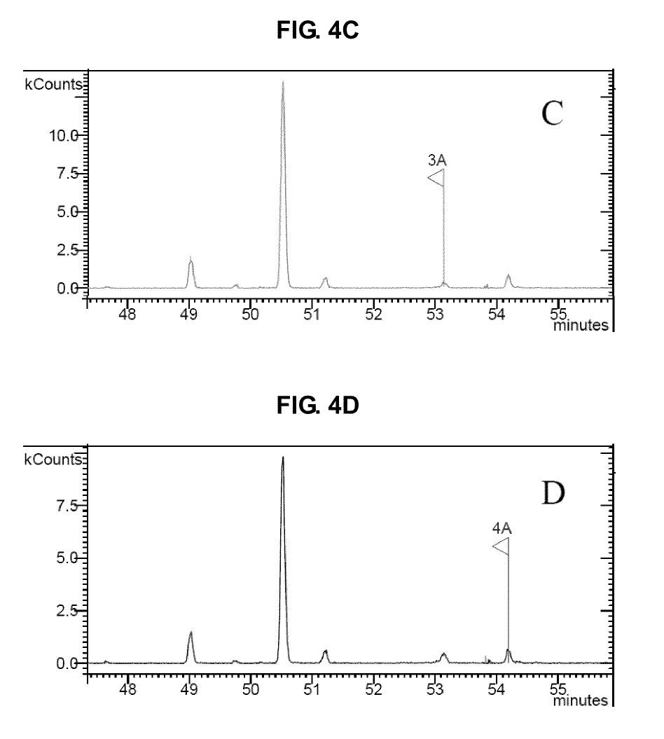

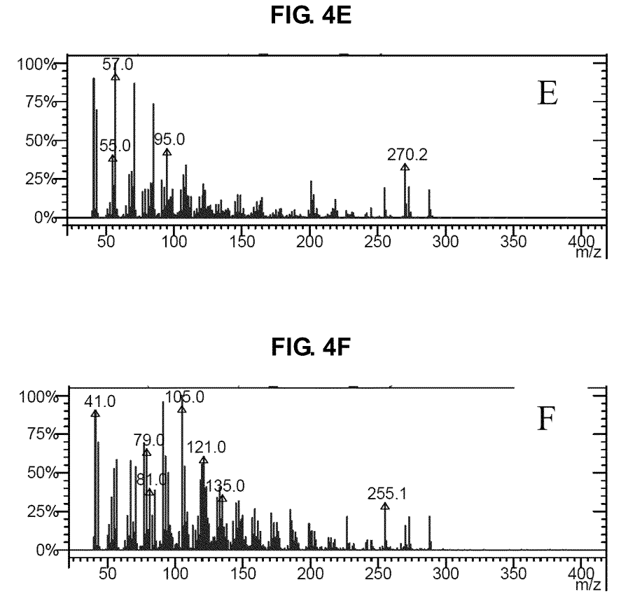

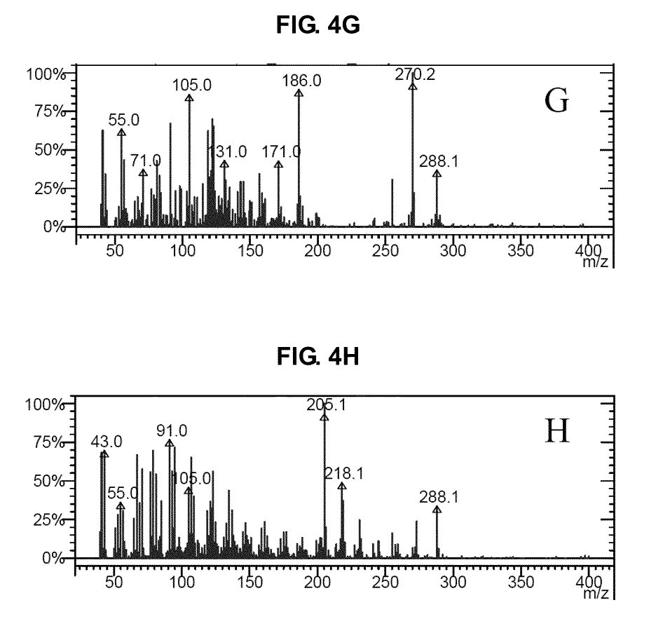

Unlike transgenic moss protonema, the major product in gametophyte cultures is not taxadien-20-ol (see e.g., peak 1A in FIG. 4A), although it is still there as revealed by the mass spectra (see e.g., FIG. 4E). The major taxane product of transgenic moss gametophytes is 5(12)-oxa-3(11)-cyclotaxane (see e.g., unlabelled peak in FIGS. 4A, 4B, 4C and 4D) based on retention times and mass spectra (data not shown). Additionally, in all of the four gametophyte cultures, other peaks were also present (see e.g., peak 2A in FIG. 4B; peak 3A in FIG. 4C; peak 4A in FIG. 4D), which have mass spectra consistent with taxadien-5-ol (see e.g., FIG. 4F), taxadien-18-ol (see e.g., FIG. 4G), and taxadien-11-ol (see e.g., FIG. 4H), respectively.

Thus is demonstrated production of taxadien-5-ol; 5(12)-oxa-3(11)-cyclotaxane; taxadien-11-ol; taxadien-18-ol; and taxadien-20-ol from transgenic moss expressing taxadiene synthase and taxadiene-5α-hydroxylase. Production of these new taxanes by transgenic P. patens did not affect moss growth, which is in contrast to the developmental delays observed in higher plants when the same enzymes have been introduced in them genetically.

### Example 4

This example describes construction of overexpression plasmids.

Methods are as described in Examples 1-2, unless otherwise indicated.

The genes for taxoid hydroxylases (namely, taxadiene-5α-hydroxylase (SEQ ID NO: 19); taxane 10β-hydroxylase (SEQ ID NO: 33); and taxane 13α-hydroxylase (SEQ ID NO: 27)) and taxadiene-5α-ol acetyltransferase (SEQ ID NO: 201) were amplified and inserted into a plasmid vector.

Each of the genes were amplified by PCR using Pfx50™ (a thermostable DNA polymerase purchased from Invitrogen) in the presence of the corresponding forward and reverse primers (see Table 2), following the manufacturer's suggested protocol.

TABLE 2
Primers for gene amplication

| | Forward Primers | Reverse Primers | Destination | |
|---|---|---|---|---|
| Gene | (5′ to 3′) | (5′ to 3′) | vector* | Promoter |
| TS | CACCATGGCTTCAGCTC | TCATACTTGAATTGGA | pTHUBIgateway | Ubiquitin |
| | TCATTTAAT | TCAATATAAACTTT | (SEQ ID NO: 213) | |
| | (SEQ ID NO: 203) | (SEQ ID NO: 204) | | |
| T5H | CACCATGGACGCCCTG | CTATGGTCTCGGAAAC | pTZ35Sgateway | 35S |
| | TATAAGAGC | AGTTTAATGG | (SEQ ID NO: 214) | |
| | (SEQ ID NO: 205) | (SEQ ID NO: 206) | | |
| T10H | CACCATGGATAGCTTCA | TTAGGATCTCGGAAAA | pTN2X35Sgateway | 2 × 355 |
| | TTTTTCTGA | AGTTTTATGG | (SEQ ID NO: 215) | |
| | (SEQ ID NO: 207) | (SEQ ID NO: 208) | | |
| T13H | CACCATGGATGCCCTTA | TTAAGATCTGGAATAG | pTN2X35Sgateway | 2 × 355 |
| | AGCAATTGG | AGTTTAATGG | (SEQ ID NO: 215) | |
| | (SEQ ID NO: 209) | (SEQ ID NO: 210) | | |
| TAT | CACCATGGAGAAGACA | TCATACTTTAGCCACA | pTN2X35Sgateway | 2 × 355 |
| | GATTTACACG | TATTTTTTCAT | (SEQ ID NO: 215) | |
| | (SEQ ID NO: 211) | (SEQ ID NO: 212) | | |

The PCR products were cloned into pENTR entry vector using the TOPO Cloning kit of Invitrogen, following manufacturer's protocol. Each of the genes were then transferred to the destination vectors specified above using LR Clonase II (also from Invitrogen), following the manufacturer's protocol. This method was described in Transgenic Research (2009) 18, 655-660.

The integrity of the plasmids were verified by PCR, restriction digests and sequencing.

What is claimed is:

1. A moss cell comprising:
at least one heterologous nucleic acid molecule encoding a polypeptide having a diterpenoid biosynthetic activity;
at least one promoter functional in a moss cell; and
at least one transcriptional termination sequence;
wherein
the promoter, the at least one heterologous nucleic acid molecule, and the transcriptional termination sequence are operably associated in the 5′ to 3′ direction of transcription;
the polypeptide having a diterpenoid biosynthetic activity is selected from the group consisting of taxadiene synthase; taxadiene-5α-hydroxylase; taxadiene-5α-ol-acetyl transferase; taxane-13α-hydroxylase; taxane-10β-hydroxylase; taxoid 14β-hydroxylase; taxoid-9α-hydroxylase; taxoid-2α-hydroxylase; taxoid-7β-hydroxylase; 2α-hydroxytaxane 2-O-benzoyl transferase (i.e., taxoid-2α-O-benzoyl transferase); taxoid C1β-hydroxylase; taxoid C4β, C20-epoxidase; abietadiene synthase; abietadienol/abietadienal oxidase; ent-copalyl diphosphate synthase; ent-Kaurene synthase; ent-Kaurene oxidase; kaurenoic acid 13-hydroxylase; UDP-glycosyltransferase (UGT) converting steviolmonoside to steviolbioside; UGT85C2; UGT74G1; UGT76G1; CBT-ol cyclase; CYP71D16; syn-copalyl diphosphate synthase; syn-pimaradiene synthase; ent-sandaracopimaradiene synthase; syn-stemarene synthase; and ent-cassadiene synthase;
expression of the heterologous nucleic acid molecule in the cell results in production of at least one terpenoid compound selected from the group consisting of taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin III; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.

2. The moss cell of claim 1 comprising:
a first heterologous nucleic acid molecule comprising a nucleotide sequence encoding a taxadiene synthase;
wherein,
the nucleotide sequence encoding taxadiene synthase is selected from the group consisting of
(i) a nucleotide sequence encoding a taxadiene synthase selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17, or at least about 90% sequence identity thereto and encoding a polypeptide having taxadiene synthase activity, or a complementary sequence thereto;
(ii) a nucleotide sequence encoding a polypeptide having a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, and SEQ ID NO: 18, or at least about 90% sequence identity thereto and having taxadiene synthase activity, or a complementary sequence thereto; and
(iii) an isolated polynucleotide that hybridizes under stringent conditions over the entire length of a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, and SEQ ID NO: 18, said stringent conditions comprising incubation at 65° C. in a solution comprising 6×SSC (0.9 M sodium chloride and 0.09 M sodium citrate), and which encodes a polypeptide having taxadiene synthase activity; and
the moss cell produces at least taxa-4(5),11(12)-diene.

3. The moss cell of claim 2 comprising:
a second heterologous nucleic acid molecule comprising a polynucleotide encoding a taxadiene 5α-hydroxylase;
wherein,
the nucleotide sequence encoding taxadiene 5α-hydroxylase is selected from the group consisting of
(i) a nucleotide sequence of SEQ ID NO: 19 or SEQ ID NO: 21, or at least about 90% sequence identity thereto and encoding a polypeptide having taxadiene 5a-hydroxylase activity, or a complementary sequence thereto;
(ii) a nucleotide sequence encoding a polypeptide having SEQ ID NO: 20 or SEQ ID NO: 22, or at least about 90% sequence identity thereto and having taxadiene 5α-hydroxylase activity, or a complementary sequence thereto; and
(iii) an isolated polynucleotide that hybridizes under stringent conditions over the entire length of SEQ ID NO: 19 or SEQ ID NO: 21; said stringent conditions comprising incubation at 65° C. in a solution comprising 6×SSC (0.9 M sodium chloride and 0.09 M sodium citrate); and which encodes a polypeptide having taxadiene 5α-hydroxylase activity; and
the moss cell produces one or more compounds selected from the group consisting of taxadiene-5-ol; 5(12)-oxa-3(11)-cyclotaxane; taxadien-11-ol; taxadien-18-ol; and taxadien-20-ol.

4. The moss cell of claim 3 comprising:
a third heterologous nucleic acid molecule comprising a polynucleotide encoding a taxane-13α-hydroxylase;
wherein,
the polynucleotide encoding taxane-13α-hydroxylase is selected from the group consisting of
(i) a nucleotide sequence of SEQ ID NO: 27, SEQ ID NO: 29, or SEQ ID NO: 31, or at least about 90% sequence identity thereto and encoding a polypeptide having taxane-13α-hydroxylase activity, or a complementary sequence thereto;
(ii) a nucleotide sequence encoding a polypeptide having SEQ ID NO: 28, SEQ ID NO: 30, or SEQ ID NO: 32, or at least about 90% sequence identity thereto and having taxane-13α-hydroxylase activity, or a complementary sequence thereto; and
(iii) an isolated polynucleotide that hybridizes under stringent conditions thereto over the entire length of SEQ ID NO: 27, SEQ ID NO: 29, or SEQ ID NO: 31, said stringent conditions comprising incubation at 65° C. in a solution comprising 6×SSC (0.9 M sodium chloride and 0.09 M sodium citrate), and which encodes a polypeptide having taxane-13α-hydroxylase activity; and
the moss cell produces at least taxadiene-5α,13α-diol.

5. The moss cell of claim 3 comprising:
a third heterologous nucleic acid molecule comprising a polynucleotide encoding a taxadiene-5α-ol-acetyl transferase;
wherein,
the polynucleotide encoding taxadiene-5α-ol-acetyl transferase is selected from the group consisting of
(i) a nucleotide sequence of SEQ ID NO: 23, SEQ ID NO: 25, or SEQ ID NO: 201, or at least about 90% sequence identity thereto and encoding a polypeptide having taxadiene-5α-ol-acetyl transferase activity, or a complementary sequence thereto;
(ii) a nucleotide sequence encoding a polypeptide having SEQ ID NO: 24, SEQ ID NO: 26, or SEQ ID NO: 202, or at least about 90% sequence identity thereto and having taxadiene-5α-ol-acetyl transferase activity, or a complementary sequence thereto; and
(iii) an isolated polynucleotide that hybridizes under stringent conditions thereto over the entire length of SEQ ID NO: 23, SEQ ID NO: 25, or SEQ ID NO: 201, said stringent conditions comprising incubation at 65° C. in a solution comprising 6×SSC (0.9 M sodium chloride and 0.09 M sodium citrate), and which encodes a polypeptide having taxadiene-5α-ol-acetyl transferase activity; and
the moss cell produces at least 5α-acetoxy-taxadiene.

6. The moss cell of claim 5 comprising:
a fourth heterologous nucleic acid molecule comprising a polynucleotide encoding a taxane-10β-hydroxylase;
wherein,
the polynucleotide encoding taxane-10β-hydroxylase is selected from the group consisting of
(i) a nucleotide sequence of SEQ ID NO: 33, SEQ ID NO: 35, SEQ ID NO: 37, or SEQ ID NO: 39, or at least about 90% sequence identity thereto and encoding a polypeptide having taxane-10β-hydroxylase activity, or a complementary sequence thereto;
(ii) a nucleotide sequence encoding a polypeptide having SEQ ID NO: 34, SEQ ID NO: 36, SEQ ID NO: 38, or SEQ ID NO: 40, or at least about 90% sequence identity thereto and having taxane-10β-hydroxylase activity, or a complementary sequence thereto; and
(iii) an isolated polynucleotide that hybridizes under stringent conditions thereto over the entire length of SEQ ID NO: 33, SEQ ID NO: 35, SEQ ID NO: 37, or SEQ ID NO: 39, said stringent conditions comprising incubation at 65° C. in a solution comprising 6×SSC (0.9 M sodium chloride and 0.09 M sodium citrate), and which encodes a polypeptide having taxane-10β-hydroxylase activity; and
the moss cell produces at least taxadiene-5α-acetoxy-10β-ol.

7. The moss cell of claim 1 comprising:
(A) a first heterologous nucleic acid molecule comprising a nucleotide sequence encoding an abietadiene synthase, wherein the moss cell produces at least abietadiene; or
(B) a first heterologous nucleic acid molecule comprising a nucleotide sequence encoding an abietadiene synthase and a second heterologous nucleic acid molecule comprising a nucleotide sequence encoding an abietadienol/abietadienal oxidase, wherein the moss cell produces at least abietic acid;
wherein,
(i) the polynucleotide encoding abietadiene synthase is selected from the group consisting of
(a) a nucleotide sequence of SEQ ID NO: 61, SEQ ID NO: 63, SEQ ID NO: 65, or SEQ ID NO: 67, or at least about 90% sequence identity thereto and encoding a polypeptide having abietadiene synthase activity, or a complementary sequence thereto;
(b) a nucleotide sequence encoding a polypeptide having SEQ ID NO: 62, SEQ ID NO: 64, SEQ ID NO: 66, or SEQ ID NO: 68, or at least about 90% sequence identity thereto and having abietadiene synthase activity, or a complementary sequence thereto; and
(c) an isolated polynucleotide that hybridizes under stringent conditions thereto over the entire length of SEQ ID NO: 61, SEQ ID NO: 63, SEQ ID NO: 65, or SEQ ID NO: 67, said stringent conditions comprising incubation at 65° C. in a solution comprising 6×SSC (0.9 M sodium chloride and 0.09 M sodium citrate), and which encodes a polypeptide having abietadiene synthase activity; and
(ii) the polynucleotide encoding abietadienol/abietadienal oxidase is selected from the group consisting of
(a) a nucleotide sequence of SEQ ID NO: 69, or at least about 90% sequence identity thereto and encoding a polypeptide having abietadienol/abietadienal oxidase activity, or a complementary sequence thereto;
(b) a nucleotide sequence encoding a polypeptide having SEQ ID NO: 70, or at least about 90% sequence identity thereto and having abietadienol/abietadienal oxidase activity, or a complementary sequence thereto; and
(c) an isolated polynucleotide that hybridizes under stringent conditions thereto over the entire length of SEQ ID NO: 69, said stringent conditions comprising incubation at 65° C. in a solution comprising 6×SSC (0.9 M sodium chloride and 0.09 M sodium citrate), and which encodes a polypeptide having abietadienol/abietadienal oxidase activity.

8. The moss cell of claim 1 comprising:
(A) a first heterologous nucleic acid molecule comprising a nucleotide sequence encoding an ent-copalyl diphosphate synthase;
a second heterologous nucleic acid molecule comprising a nucleotide sequence encoding an ent-Kaurene synthase; and
a third heterologous nucleic acid molecule comprising a nucleotide sequence encoding an ent-Kaurene oxidase;
wherein the moss cell produces at least kaurenoic acid;
(B) a first heterologous nucleic acid molecule comprising a nucleotide sequence encoding an ent-copalyl diphosphate synthase;
a second heterologous nucleic acid molecule comprising a nucleotide sequence encoding an ent-Kaurene synthase;
a third heterologous nucleic acid molecule comprising a nucleotide sequence encoding an ent-Kaurene oxidase; and
a fourth heterologous nucleic acid molecule comprising a nucleotide sequence encoding an kaurenoic acid 13-hydroxylase;
wherein the moss cell produces at least steviol;
(C) a first heterologous nucleic acid molecule comprising a nucleotide sequence encoding an ent-copalyl diphosphate synthase;
a second heterologous nucleic acid molecule comprising a nucleotide sequence encoding an ent-Kaurene synthase;
a third heterologous nucleic acid molecule comprising a nucleotide sequence encoding an ent-Kaurene oxidase; and
a fourth heterologous nucleic acid molecule comprising a nucleotide sequence encoding an kaurenoic acid 13-hydroxylase; and
a fifth heterologous nucleic acid molecule comprising a nucleotide sequence encoding an UDP-glycosyltransferase (UGT) UGT85C2;
wherein the moss cell produces at least steviolmonoside;
(D) a first heterologous nucleic acid molecule comprising a nucleotide sequence encoding an ent-copalyl diphosphate synthase;
a second heterologous nucleic acid molecule comprising a nucleotide sequence encoding an ent-Kaurene synthase;
a third heterologous nucleic acid molecule comprising a nucleotide sequence encoding an ent-Kaurene oxidase; and
a fourth heterologous nucleic acid molecule comprising a nucleotide sequence encoding an kaurenoic acid 13-hydroxylase;
a fifth heterologous nucleic acid molecule comprising a nucleotide sequence encoding an UDP-glycosyltransferase (UGT) UGT85C2; and
a sixth heterologous nucleic acid molecule comprising a nucleotide sequence encoding a UGT74G1;
wherein the moss cell produces at least stevioside; or
(E) a first heterologous nucleic acid molecule comprising a nucleotide sequence encoding an ent-copalyl diphosphate synthase;
a second heterologous nucleic acid molecule comprising a nucleotide sequence encoding an ent-Kaurene synthase;
a third heterologous nucleic acid molecule comprising a nucleotide sequence encoding an ent-Kaurene oxidase; and
a fourth heterologous nucleic acid molecule comprising a nucleotide sequence encoding an kaurenoic acid 13-hydroxylase;
a fifth heterologous nucleic acid molecule comprising a nucleotide sequence encoding an UDP-glycosyltransferase (UGT) UGT85C2; and
a sixth heterologous nucleic acid molecule comprising a nucleotide sequence encoding a UGT74G1; and
a seventh heterologous nucleic acid molecule comprising a nucleotide sequence encoding a UGT76G1;
wherein the moss cell produces at least rebaudioside A;
wherein
(i) the polynucleotide encoding ent-copalyl diphosphate synthase is selected from the group consisting of
(a) a nucleotide sequence of SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, or SEQ ID NO: 115, or at least about 90% sequence identity thereto and encoding a polypeptide having ent-copalyl diphosphate synthase activity, or a complementary sequence thereto;
(b) a nucleotide sequence encoding a polypeptide having SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, or SEQ ID NO: 116, or at least about 90% sequence identity thereto and having ent-copalyl diphosphate synthase activity, or a complementary sequence thereto; and
(c) an isolated polynucleotide that hybridizes under stringent conditions thereto over the entire length of SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, or SEQ ID NO: 115, said stringent conditions comprising incubation at 65° C. in a solution comprising 6×SSC (0.9 M sodium chloride and 0.09 M sodium citrate), and which encodes a polypeptide having ent-copalyl diphosphate synthase activity;
(ii) the polynucleotide encoding ent-Kaurene synthase is selected from the group consisting of
(a) a nucleotide sequence of SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, or SEQ ID NO: 143, or at least about 90% sequence identity thereto and encoding a polypeptide having ent-Kaurene synthase activity, or a complementary sequence thereto;
(b) a nucleotide sequence encoding a polypeptide having SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, or SEQ ID NO: 144, or at least about 90% sequence identity thereto and having ent-Kaurene synthase activity, or a complementary sequence thereto; and
(c) an isolated polynucleotide that hybridizes under stringent conditions thereto over the entire length of SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, or SEQ ID NO: 143, said stringent conditions comprising incubation at 65° C. in a solution comprising 6×SSC (0.9 M sodium chloride and 0.09 M sodium citrate), and which encodes a polypeptide having ent-Kaurene synthase activity; and
(iii) the polynucleotide encoding ent-Kaurene oxidase is selected from the group consisting of
(a) a nucleotide sequence of SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, or SEQ ID NO: 173, or at least about 90% sequence identity thereto and encoding a polypeptide having ent-Kaurene oxidase activity, or a complementary sequence thereto;
(b) a nucleotide sequence encoding a polypeptide having SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, or SEQ ID NO: 174, or at least about 90% sequence identity thereto and having ent-Kaurene oxidase activity, or a complementary sequence thereto; and
(c) an isolated polynucleotide that hybridizes under stringent conditions thereto over the entire length of SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, or SEQ ID NO: 173, said stringent conditions comprising incubation at 65° C. in a solution comprising 6×SSC (0.9 M sodium chloride and 0.09 M sodium citrate), and which encodes a polypeptide having ent-Kaurene oxidase activity;
(iv) the polynucleotide encoding kaurenoic acid 13-hydroxylase is selected from the group consisting of
(a) a nucleotide sequence of SEQ ID NO: 175, or at least about 90% sequence identity thereto and encoding a polypeptide having—kaurenoic acid 13-hydroxylase activity, or a complementary sequence thereto;
(b) a nucleotide sequence encoding a polypeptide having SEQ ID NO: 176, or at least about 90% sequence identity thereto and having—kaurenoic acid 13-hydroxylase activity, or a complementary sequence thereto; and
(c) an isolated polynucleotide that hybridizes under stringent conditions thereto over the entire length of SEQ ID NO: 175, said stringent conditions comprising incubation at 65° C. in a solution comprising 6×SSC (0.9 M sodium chloride and 0.09 M sodium citrate), and which encodes a polypeptide having kaurenoic acid 13-hydroxylase activity;
(v) the polynucleotide encoding UDP-glycosyltransferase (UGT) UGT85C2 is selected from the group consisting of
(a) a nucleotide sequence of SEQ ID NO: 177, or at least about 90% sequence identity thereto and encoding a polypeptide having UDP-glycosyltransferase activity, or a complementary sequence thereto;
(b) a nucleotide sequence encoding a polypeptide having SEQ ID NO: 178, or at least about 90% sequence identity thereto and having—UDP-glycosyltransferase activity, or a complementary sequence thereto; and
(c) an isolated polynucleotide that hybridizes under stringent conditions thereto over the entire length of SEQ ID NO: 177, said stringent conditions comprising incubation at 65° C. in a solution comprising 6×SSC (0.9 M sodium chloride and 0.09 M sodium citrate), and which encodes a polypeptide having UDP-glycosyltransferase activity; and
(vi) the polynucleotide encoding UGT74G1 is selected from the group consisting of
(a) a nucleotide sequence of SEQ ID NO: 179, or at least about 90% sequence identity thereto and encoding a polypeptide having UDP-glycosyltransferase activity, or a complementary sequence thereto;
(b) a nucleotide sequence encoding a polypeptide having SEQ ID NO: 180, or at least about 90% sequence identity thereto and having—UDP-glycosyltransferase activity, or a complementary sequence thereto; and
(c) an isolated polynucleotide that hybridizes under stringent conditions thereto over the entire length of SEQ ID NO: 179, said stringent conditions comprising incubation at 65° C. in a solution comprising 6×SSC (0.9 M sodium chloride and 0.09 M sodium citrate), and which encodes a polypeptide having UDP-glycosyltransferase activity; and
(vii) the polynucleotide encoding UGT76G1 is selected from the group consisting of
(a) a nucleotide sequence of SEQ ID NO: 181, or at least about 90% sequence identity thereto and encoding a polypeptide having UDP-glycosyltransferase activity, or a complementary sequence thereto;
(b) a nucleotide sequence encoding a polypeptide having SEQ ID NO: 182, or at least about 90% sequence identity thereto and having UDP-glycosyltransferase activity, or a complementary sequence thereto; and
(c) an isolated polynucleotide that hybridizes under stringent conditions thereto over the entire length of SEQ ID NO: 181, said stringent conditions comprising incubation at 65° C. in a solution comprising 6×SSC (0.9 M sodium chloride and 0.09 M sodium citrate), and which encodes a polypeptide having UDP-glycosyltransferase activity.

9. The moss cell of claim 1 comprising:
a first heterologous nucleic acid molecule comprising a nucleotide sequence encoding a CBT-ol cyclase; and
a second heterologous nucleic acid molecule comprising a nucleotide sequence encoding a CYP71D16;
wherein
(A) the polynucleotide encoding CBT-ol cyclase is selected from the group consisting of
(i) a nucleotide sequence of SEQ ID NO: 183 or SEQ ID NO: 185, or at least about 90% sequence identity thereto and encoding a polypeptide having CBT-ol cyclase activity, or a complementary sequence thereto;
(ii) a nucleotide sequence encoding a polypeptide having SEQ ID NO: 184 or SEQ ID NO: 186, or at least about 90% sequence identity thereto and having CBT-ol cyclase activity, or a complementary sequence thereto; and
(iii) an isolated polynucleotide that hybridizes under stringent conditions thereto over the entire length of SEQ ID NO: 183 or SEQ ID NO: 185, said stringent conditions comprising incubation at 65° C. in a solution comprising 6×SSC (0.9 M sodium chloride and 0.09 M sodium citrate), and which encodes a polypeptide having CBT-ol cyclase activity; and
(B) the polynucleotide encoding CYP71D16 is selected from the group consisting of
(i) a nucleotide sequence of SEQ ID NO: 187, or at least about 90% sequence identity thereto and encoding a polypeptide having CYP71D16 activity, or a complementary sequence thereto;
(ii) a nucleotide sequence encoding a polypeptide having SEQ ID NO: 188, or at least about 90% sequence identity thereto and having CYP71D16 activity, or a complementary sequence thereto; and
(iii) an isolated polynucleotide that hybridizes under stringent conditions thereto over the entire length of SEQ ID NO: 187, said stringent conditions comprising incubation at 65° C. in a solution comprising 6×SSC (0.9 M sodium chloride and 0.09 M sodium citrate), and which encodes a polypeptide having CYP71D16 activity; and
the moss cell produces at least a cembranoid.

10. The moss cell of claim 1 comprising:
a first heterologous nucleic acid molecule comprising a nucleotide sequence encoding a syn-copalyl diphosphate synthase; and
a second heterologous nucleic acid molecule comprising a nucleotide sequence encoding a syn-pimaradiene synthase;
wherein
(A) the polynucleotide encoding syn-copalyl diphosphate synthase is selected from the group consisting of
(i) a nucleotide sequence of SEQ ID NO: 189, or at least about 90% sequence identity thereto and encoding a polypeptide having syn-copalyl diphosphate synthase activity, or a complementary sequence thereto;
(ii) a nucleotide sequence encoding a polypeptide having SEQ ID NO: 190, or at least about 90% sequence identity thereto and having—syn-copalyldiphosphate synthase activity, or a complementary sequence thereto; and
(iii) an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having syn-copalyl diphosphate synthase activity; and
(B) the polynucleotide encoding syn-pimaradiene synthase is selected from the group consisting of
(i) a nucleotide sequence of SEQ ID NO: 191 or SEQ ID NO: 193, or at least about 90% sequence identity thereto and encoding a polypeptide having syn-pimaradiene synthase activity, or a complementary sequence thereto;
(ii) a nucleotide sequence encoding a polypeptide having SEQ ID NO: 192 or SEQ ID NO: 194, or at least about 90% sequence identity thereto and having syn-pimaradiene synthase activity, or a complementary sequence thereto; and
(iii) an isolated polynucleotide that hybridizes under stringent conditions thereto over the entire length of SEQ ID NO: 191 or SEQ ID NO: 193, said stringent conditions comprising incubation at 65° C. in a solution comprising 6×SSC (0.9 M sodium chloride and 0.09 M sodium citrate), and which encodes a polypeptide having syn-pimaradiene synthase activity; and
the moss cell produces at least momilactone A or momilactone B.

11. The moss cell of claim 1 comprising:
a first heterologous nucleic acid molecule comprising a nucleotide sequence encoding a ent-Copalyl diphosphate synthase; and
a second heterologous nucleic acid molecule comprising a nucleotide sequence encoding a ent-sandaracopimaradiene synthase;
wherein
(A) the polynucleotide encoding ent-Copalyl diphosphate synthase is selected from the group consisting of
(i) a nucleotide sequence of SEQ ID NO: 189, or at least about 90% sequence identity thereto and encoding a polypeptide having ent-copalyl diphosphate synthase activity, or a complementary sequence thereto;
(ii) a nucleotide sequence encoding a polypeptide having SEQ ID NO: 190, or at least about 90% sequence identity thereto and having—ent-copalyldiphosphate synthase activity, or a complementary sequence thereto; and
(iii) an isolated polynucleotide that hybridizes under stringent conditions thereto over the entire length of SEQ ID NO: 189, said stringent conditions comprising incubation at 65° C. in a solution comprising 6×SSC (0.9 M sodium chloride and 0.09 M sodium citrate), and which encodes a polypeptide having ent-copalyl diphosphate synthase activity; and
(B) the polynucleotide encoding ent-sandaracopimaradiene synthase is selected from the group consisting of
(i) a nucleotide sequence of SEQ ID NO: 195, or at least about 90% sequence identity thereto and encoding a polypeptide having ent-sandaracopimaradiene synthase activity, or a complementary sequence thereto;
(ii) a nucleotide sequence encoding a polypeptide having SEQ ID NO: 196, or at least about 90% sequence identity thereto and having ent-sandaracopimaradiene synthase activity, or a complementary sequence thereto; and
(iii) an isolated polynucleotide that hybridizes under stringent conditions thereto over the entire length of SEQ ID NO: 195, said stringent conditions comprising incubation at 65° C. in a solution comprising 6×SSC (0.9 M sodium chloride and 0.09 M sodium citrate), and which encodes a polypeptide having ent-sandaracopimaradiene synthase activity; and
the moss cell produces at least one of oryzalexins A, B, C, D, E, or F.

12. The moss cell of claim 1 comprising:
a first heterologous nucleic acid molecule comprising a nucleotide sequence encoding a syn-copalyl diphosphate synthase; and
a second heterologous nucleic acid molecule comprising a nucleotide sequence encoding a syn-stemarene synthase;
wherein
(A) the polynucleotide encoding syn-copalyl diphosphate synthase is selected from the group consisting of
(i) a nucleotide sequence of SEQ ID NO: 189, or at least about 90% sequence identity thereto and encoding a polypeptide having syn-copalyl diphosphate synthase activity, or a complementary sequence thereto;
(ii) a nucleotide sequence encoding a polypeptide having SEQ ID NO: 190, or at least about 90% sequence identity thereto and having—syn-copalyldiphosphate synthase activity, or a complementary sequence thereto; and
(iii) an isolated polynucleotide that hybridizes under stringent conditions thereto over the entire length of said sequence; said stringent conditions comprising incubation at 65° C. in a solution comprising 6×SSC (0.9 M sodium chloride and 0.09 M sodium citrate); and which encodes a polypeptide having syn-copalyl diphosphate synthase activity; and
(B) the polynucleotide encoding syn-stemarene synthase is selected from the group consisting of
(i) a nucleotide sequence of SEQ ID NO: 197, or at least about 90% sequence identity thereto and encoding a polypeptide having syn-stemarene synthase activity, or a complementary sequence thereto;
(ii) a nucleotide sequence encoding a polypeptide having SEQ ID NO: 198, or at least about 90% sequence identity thereto and having syn-stemarene synthase activity, or a complementary sequence thereto; and
(iii) an isolated polynucleotide that hybridizes under stringent conditions thereto over the entire length of SEQ ID NO: 197, said stringent conditions comprising incubation at 65° C. in a solution comprising 6×SSC (0.9 M sodium chloride and 0.09 M sodium citrate), and which encodes a polypeptide having syn-stemarene synthase activity; and
the moss cell produces at least oryzalexin S.

13. The moss cell of claim 1 comprising:
a first heterologous nucleic acid molecule comprising a nucleotide sequence encoding a ent-Copalyl diphosphate synthase; and
a second heterologous nucleic acid molecule comprising a nucleotide sequence encoding a ent-cassadiene synthase;
wherein,
(A) the polynucleotide encoding ent-Copalyl diphosphate synthase is selected from the group consisting of
(i) a nucleotide sequence of SEQ ID NO: 189, or at least about 90% sequence identity thereto and encoding a polypeptide having ent-copalyl diphosphate synthase activity, or a complementary sequence thereto;
(ii) a nucleotide sequence encoding a polypeptide having SEQ ID NO: 190, or at least about 90% sequence identity thereto and having—ent-copalyldiphosphate synthase activity, or a complementary sequence thereto; and
(iii) an isolated polynucleotide that hybridizes under stringent conditions thereto over the entire length of SEQ ID NO: 189, said stringent conditions comprising incubation at 65° C. in a solution comprising 6×SSC (0.9 M sodium chloride and 0.09 M sodium citrate), and which encodes a polypeptide having ent-copalyl diphosphate synthase activity; and
(B) the polynucleotide encoding ent-cassadiene synthase is selected from the group consisting of
(i) a nucleotide sequence of SEQ ID NO: 199, or at least about 90% sequence identity thereto and encoding a polypeptide having ent-cassadiene synthase activity, or a complementary sequence thereto;
(ii) a nucleotide sequence encoding a polypeptide having SEQ ID NO: 200, or at least about 90% sequence identity thereto having ent-cassadiene synthase activity, or a complementary sequence thereto; and
(iii) an isolated polynucleotide that hybridizes under stringent conditions thereto over the entire length of SEQ ID NO: 199, said stringent conditions comprising incubation at 65° C. in a solution comprising 6×SSC (0.9 M sodium chloride and 0.09 M sodium citrate), and which encodes a polypeptide having ent-cassadiene synthase activity; and
the moss cell produces at least one of phytocassanes A-E.

14. The moss cell of claim 1, wherein
(i) expression or activity is reduced or eliminated for one or more of Mevalonate diphosphate decarboxylase; Mevalonate kinase; HMG-CoA reductase; Squalene epoxidase; 4-Hydroxyphenylpyruvate dioxygenase; Geranylgeranyl pyrophosphate synthase; ent-Kaurene synthetase; Chorismate mutase; Farnesyl pyrophosphate synthase; Phytoene synthase; Adenylate isopentenyltransferase; Squalene-hopene-cyclase; γ-Tocopherol methyltransferase; Geranylgeranyl reductase; Phytoene desaturase; ζ-Carotene desaturase; Geranylgeranyltransferase I; Zeaxanthin epoxidase; Copalyl diphosphate synthase; 2-Heptaprenyl-1,4-naphthoquinone methyltransferase; 9-cis-Epoxycarotenoid cleavage dioxygenase; 1-Deoxy-D-xylulose 5-phosphate synthase; Lycopene ε cyclase; and 2-Methyl-6-phytylhydroquinone 3-methyltransferase; or
(ii) expression or activity of diterpene synthase or kaurene synthase is reduced or eliminated and the moss cell produces decreased levels of ent-kaurene or 16-hydroxykaurane compared to a moss cell not comprising the DNA construct.

15. A method of producing a terpenoid compound comprising culturing the moss cell of claim 1.

16. A method of producing a moss cell according to claim 1 comprising:
introducing into the moss cell at least a first heterologous nucleic acid molecule;
wherein expression of the heterologous nucleic acid molecule in the cell results in production of at least one terpenoid compound selected from the group consisting of taxa-4(5),11(12)-diene; taxa-4(20),11(12)-diene; taxa-3(4),11(12)-diene; verticillene; taxadiene-5-ol; 5(12)-oxa-3(11)-cyclotaxane; taxadien-11-ol; taxadien-18-ol; taxadien-20-ol; 5α-hydroxy-taxa-4(20),11(12)-diene; 5α,13α-dihydroxy-taxa-4(20),11(12)-diene; 5α-acetoxy-taxa-4(20),11(12)-diene; 5α-acetoxy-10β-hydroxy-taxa-4(20),11(12)-diene; 5α-acetoxy-10β,14β-dihydroxy-taxadiene; taxadiene-5α-acetoxy-13β-ol; taxadiene-5α,13α-diol; taxadiene-5α-acetoxy-10β-ol; baccatin Ill; 10-deacetylbaccatin III; abietadiene; abietic acid; steviol; steviolmonoside; stevioside; rebaudioside A; forskolin; sclareol; kaurenoic acid; a cembranoid; momilactone A; momilactone B; oryzalexins A-F; oryzalexin S; and phytocassanes A-E.
